(19) United States
(12) Patent Application Publication
(10) Pub. No.: US 2016/0196666 A1
(43) Pub. Date: Jul. 7, 2016

(54) SYSTEMS FOR DETECTING AND TRACKING OF OBJECTS AND CO-REGISTRATION

(71) Applicant: ANGIOMETRIX CORPORATION, Bethesda, MD (US)

(72) Inventors: Vikram VENKATRAGHAVAN, Bangalore (IN); Raghavan SUBRAMANIYAN, Bangalore (IN); Goutam DUTTA, Bangalore (IN)

(73) Assignee: ANGIOMETRIX CORPORATION, Bethesda, MD (US)

(21) Appl. No.: 14/823,635

(22) Filed: Aug. 11, 2015

Related U.S. Application Data
(63) Continuation of application No. PCT/US2014/015836, filed on Feb. 11, 2014.
(60) Provisional application No. 61/763,275, filed on Feb. 11, 2013.
(60) Provisional application No. 61/872,741, filed on Sep. 1, 2013.
(60) Provisional application No. 61/914,463, filed on Dec. 11, 2013.

(51) Int. Cl. G06T 7/20; A61B 6/00; A61K 49/04; G06T 11/00; G06T 7/00; A61B 5/06; A61B 5/00

(52) U.S. Cl. CPC G06T 7/2053; A61B 5/065; A61B 6/5264; A61B 5/7207; G06T 11/008; G06T 7/0042; A61K 49/04; G06T 2207/10064; G06T 2207/20224; G06T 2207/30204; G06T 2207/30048; G06T 2207/30061; G06T 2207/20104; G06T 2207/30101

(57) ABSTRACT

Systems for detecting and tracking of objects and co-registration are described which utilizes methods to create a linearized view of a lumen using multiple imaged frames. In reality a lumen has a trajectory in 3-D, but only a 2-D projected view is available for viewing. The linearized view unravels this 3-D trajectory thus creating a linearized map for every point on the lumen trajectory as seen on the 2-D display. In one mode of the invention, the trajectory is represented as a linearized display along 1 dimension. This linearized view is also combined with lumen measurement data and the result is displayed concurrently on a single image. In another mode of the invention, the position of a treatment device is displayed on the linearized map in real time. In a further extension of this mode, the profile of the lumen dimension is also displayed on this linearized map.

### CROSS-REFERENCE TO RELATED APPLICATIONS

This application is a continuation of PCT International Patent Appl. No. PCT/US2014/015836 filed Feb. 11, 2014, which claims the benefit of priority to U.S. Prov. Apps. 61/763,275 filed Feb. 11, 2013; 61/872,741 filed Sep. 1, 2013; and 61/914,463 filed Dec. 11, 2013, each of which is incorporated herein by reference in its entirety.

### FIELD OF THE INVENTION

The invention relates generally to intravascular medical devices. More particularly, the invention relates to guidewires, catheters, and related devices which are introduced intravascularly and utilized for obtaining various physiological parameters and processing them for mapping of various lumens.

### BACKGROUND OF THE INVENTION

There are several devices such as IVUS and OCT wires or catheters that measure dimensions of lumens. These devices are inserted into the lumen to the end of or just past the region of interest. The device is then pulled back using a stepper motor while lumen measurements are made. This allows for creating a “linear” map of lumen dimension along the lumen. In a typical representation, the X axis of the map would be the distance of the measurement point from a reference point, and the Y axis would be the corresponding lumen dimension (e.g. cross sectional area or diameter). This allows the physician to ascertain the length, cross-sectional area and profile of a lesion (diseased portion of a blood vessel). Both, the length and cross-sectional area of a lesion are desirable to determine the severity of the lesion as well as the potential treatment plan. For example, if a stent is to be deployed, the diameter of the stent is determined by the measured diameter in the neighboring un-diseased portion of the blood vessel. The length of the stent would be determined by the length of significantly diseased section of the blood vessel.

While IVUS and OCT give a good estimate of the length and cross-sectional area of a lesion, one problem is that when the treatment is delivered it does not preserve position. After measurement is made, the measurement device is retracted and the treatment device is introduced into the lumen. There is no existing mechanism to determine if the stent is positioned correctly at the diseased site. The “linear” map created during measurement is available, but the current position of the stent in this linear map is not available. In other words the obtained information is not co-registered with the X-ray image.

The other problem is that the primary display used by physicians to view the X-ray images during diagnosis and treatment is typically a 2-D image taken from a certain angle and with a certain zoom factor. Since these images are a projection of a structure that is essentially 3-D in nature, the apparent length and trajectory would be a distorted version of the truth. For example, a segment of a blood vessel that is 20 mm, the apparent length of the segment depends on the viewing angle. If the segment is in the image plane, it would appear to be of a certain length. If it subtends an angle to the image plane, it appears shorter. This makes it difficult to accurately judge actual lengths from an X-ray image. Moreover, in a quasi-periodic motion of a moving organ, different phases during the motion correspond to different structure of lumen in 3-D. This in turn corresponds to different 2-D projections in each phase of the quasi-periodic motion. Thus creating a linearized and co-registered map of a lumen can either be done for each phase of the motion separately or it can be done for a chosen representative phase. In case of the latter, mapping of the 2-D projection of lumen from each individual phase to the representative phase is needed. Even in this latter case, it is not practically possible to avoid the need for motion compensation even if the 2-D projection from a chosen phase of the heartbeat is used. There are several reasons for this. Firstly, the contribution to motion also comes from other reasons such as breathing. This motion also needs to be accounted for. Secondly, because images are captured at discrete points in time (e.g., at 15 frames per second), there may not be a frame available at precise time instance of a particular phase of the heartbeat. Choosing the nearest frame would leave behind a residual motion that can be significant and would need to be compensated for. Thirdly, choosing only one phase of the heartbeat causes a large time gap between two successive frames chosen for a particular phase of the heartbeat. For example, if the heart rate is 60 beats per minute, only one frame per second would be used for processing (typically images are available at 15 or 30 frames per second). This would make it very difficult to track moving markers on the device.

Accordingly, a system that allows co-registering of measured lumen dimensions with a position of the treatment device and details about how the linear map is created with multiple imaged frames and further details about co-registrations is desired.

### SUMMARY OF THE INVENTION

Disclosed are efficient methods to create a linearized view of a body lumen with the help of multiple image frames. In reality a lumen has a trajectory in 3-D, but only a 2-D projected view is available for viewing. The linearized view unravels this 3-D trajectory thus creating a linearized map for every point on the lumen trajectory as seen on the 2-D display. In one mode of the invention, the trajectory is represented as a linearized display along 1-dimension. This linearized view is also combined with lumen measurement data and the result is displayed concurrently on a single image referred to as the analysis mode. This mode of operation can assist an interventionalist in uniquely demarcating a lesion, if there are any, and identify its position. Analysis mode of operation also helps in linearizing the blood vessel while an endo-lumen device is inserted in it (or manually pulled back) as opposed to the popularly used technique of motorized pullback. In another mode of the invention, the position of a treatment device is displayed on the linearized map in real time referred to as the guidance mode. Additionally, the profile of the lumen dimension is also displayed on this linearized map.

Examples of devices and methods for obtaining various dimensions of lumens and which may be used with the devices, systems, and methods disclosed herein may be seen in further detail in the following: U.S. Prov. 61/383,744 filed Sep. 17, 2010; U.S. application Ser. No. 13/159,298 filed Jun. 13, 2011 (U.S. Pub. 2011/0306867); Ser. No. 13/305,610 filed Nov. 28, 2011 (U.S. Pub. 2012/0101355); Ser. No. 13/305,674 filed Nov. 28, 2011 (U.S. Pub. 2012/0101369); Ser. No. 13/305,630 filed Nov. 28, 2011 (U.S. Pub. 2012/0071782); and PCT/US2012/034557 filed Apr. 20, 2012 (designating the U.S.). Each of these applications is incorporated herein by reference in its entirety and for any purpose.

Other aspects of the invention deal with reducing the complexity of image processing that enables a real time implementation of the algorithm. In one aspect, the trajectory of an endo-lumen device is determined, and future frames use a predicted position of the device to narrow down the search range. Detection of endo lumen device and detection of radiopaque markers are combined to yield a more robust detection of each of the components and results in a more accurate linearized map. The method used to compensate for the motion of the moving organ by identifying the endo-lumen device in different phases of the motion is novel. This motion compensation in turn helps in generating a linearized and co-registered map of lumen in a representative phase of the quasi-periodic motion and in further propagating the generated map to other phases of the motion. First the two ends of the visible segment of the endo lumen device—for e.g. in a guide-wire the tip of the guide catheter and the distal coil of the guidewire—are detected. Subsequently different portions of the endo-lumen device are detected along with any radiopaque markers that may be attached to it. Another novel aspect of the invention is the mapping of the detected endo lumen segment from any or all of the previous frames to the current frame to reduce the complexity in detecting the device in subsequent frames. The detection of the endo lumen device itself is based on first detecting all possible tube like structures in the search region, and then selecting and connecting together a sub-set of such structures based on smoothness constraints to reconstruct the endo lumen device. Further, prominent structures on the guidewire are detected more reliably and are given higher weight when selecting the subset of structures. In another variant of this invention, only a subset of the endo lumen segment is detected. This is done in an incremental fashion and only the region relevant for the treatment can be detected and linearized.

Another aspect of the invention is to compensate for motion due to heartbeat and breathing, camera angle change or physical motion of the patient or the platform. The linearized view is robust to any of the aforementioned motion. This is done by using prominent structures or landmarks along the longitudinal direction of the lumen e.g. tip of the guide catheter, distal coil in a deep-seated guide wire section, stationary portion of the delineated guide-wire, stationary radiopaque markers or the farthest position of a moving radiopaque marker along the lumen under consideration, anatomical landmark such as branches along the artery. The linearized map is made with reference to these points.

Other aspects of the invention deal with reducing the complexity of image processing algorithm that enables a real time implementation of the algorithm and compensating for the periodic motion of the organ.

The image processing aspects of the innovation deals with the following:
1. Tapping the live feed video, ECG and other vital signs from the output of the imaging device.
2. Automatic selection of frames to process along with their region of interest. 13. Tracking of the endo-lumen device even in cases where orientation, position and magnification of the imaging device are altered during the procedure.
4. Quantification of biological properties of the vessel such as vessel compliance—for e.g. movement of various parts of the artery at the time of heartbeat during a cardiac intervention and vessel tortuosity (twists and turns in a vessel)
5. Selection of lesion delineators.
6. Motion compensation of the endo lumen device for computing the linearized map and for co-registration.
7. Selection of the frames where artery is being highlighted by an injected dye and using these frames for analyzing the variation of artery diameter.
8. Automatic blood vessel diameter measurement—also known as QCA—and its usage for co-registration.
9. Linearization and 3D reconstruction of lumen trajectories based on markers or end point of devices that are far apart from each other.

### BRIEF DESCRIPTION OF THE DRAWINGS

FIG. 1 shows mapping from a 2-D curve to linear representation.

FIG. 2 shows a guidewire and catheter with markers and/or Electrodes.

FIG. 3 shows a guidewire or catheter placed in a curved trajectory.

FIG. 4 shows a guide catheter and guidewire used in angioplasty.

FIG. 5 shows an illustration of the radiopaque electrodes along a guidewire inside a coronary artery.

FIG. 6 shows a block diagram illustrating various steps involved in construction of a linear map of the artery.

FIG. 7 shows a variation of the co-ordinates of the electrodes due to heart-beat.

FIG. 8 shows detection of distal coil.

FIG. 9A shows detected ends of the guidewire.

FIG. 9B shows a variation of the correlation score in the search space.

FIG. 10 shows an example of tube likeliness.

FIG. 11 shows a guidewire mapped from previous frame.

FIG. 12 show guidewire identification and refinement.

FIG. 13 shows a detected guidewire after spline fit.

FIG. 14 shows a graph indicating detection of maxima in the tube-likeliness plot taking the inherent structure of markers into consideration.

FIG. 15 shows radiopaque markers detected.

FIG. 16 shows illustration of the linearized path co-registered with the lumen diameter and cross sectional area information measured near a stenosis.

FIG. 17 shows a display of position of catheter on linear map.

FIG. 18 shows a block diagram enlisting various modules along with the output it provides to the end user.

FIG. 19 shows variation of SSD with respect to time (or frames).

FIG. 20 shows a histogram of SSD.

FIG. 21 shows a capture image.

FIG. 22 shows a directional tube-likeliness metric overlaid on an original image (shown at 5× magnification).

FIG. 23 show consecutive frames during an X-ray angiography procedure captured at the time of linear translation of a C-arm machine.

FIG. 24 shows a variation of SSD for various possible values of translation.

FIG. 25 shows a detected guidewire.

FIG. 26 shows an example of a self-loop in a guidewire.

FIG. 27 shows a block diagram of a marker detection algorithm.

FIG. 28 shows a block diagram of a linearization algorithm.

FIG. 29 shows a block diagram illustrating a typical system used for linear mapping.

FIG. 30 shows an example of lumen trajectory variations across a heart cycle.

FIG. 31 shows an example of lumen trajectories mapped across two phases of the heart cycle.

FIG. 32 shows a block diagram illustrating a particular system used for linear mapping.

FIG. 33 shows an X-ray image illustrating the visible distal section of a guidewire.

FIGS. 34-1 to 34-6 show how a guidewire may traverse the entirety of a region of interest in an artery before reaching its final position.

FIG. 35 shows an X-ray image of a guidewire having its tip section readily identified.

FIG. 36 shows an example of a balloon catheter having two markers spaced apart by a known distance.

FIG. 37 show a guidewire over two successive frames as it translates along the trajectory of a blood vessel.

FIG. 38 shows a guidewire as it moves through different points on the trajectory over successive frames.

FIGS. 39 and 40 shows how the tip section of a guidewire moves between each frame and its relationship with pixel distances.

FIGS. 41A and 41B show images with CABG wires and interventional tools in two different phases of the heartbeat.

FIG. 42 shows an illustration of the 5 degrees of freedom of a C-arm machine.

FIGS. 43A to 43C an angiogram and identified highlighted skeleton of an artery of interest.

FIG. 44 show illustrations of the process of highlighting a blood vessel through injecting a dye.

FIG. 45 shows the skeletonization of the blood vessel path.

FIGS. 46A and 46B show angiograms which highlight the artery of interest.

FIG. 47 shows a block diagram of an automatic QCA algorithm.

FIG. 48 shows a block diagram of a fly-through view generation algorithm.

FIG. 49 shows a block diagram of various algorithms involved in the analysis mode of operation.

FIGS. 50A and 50B show examples for demarcating a lesion which is tehn superimposed on a static angiogram and live angiogram.

### DETAILED DESCRIPTION OF THE INVENTION

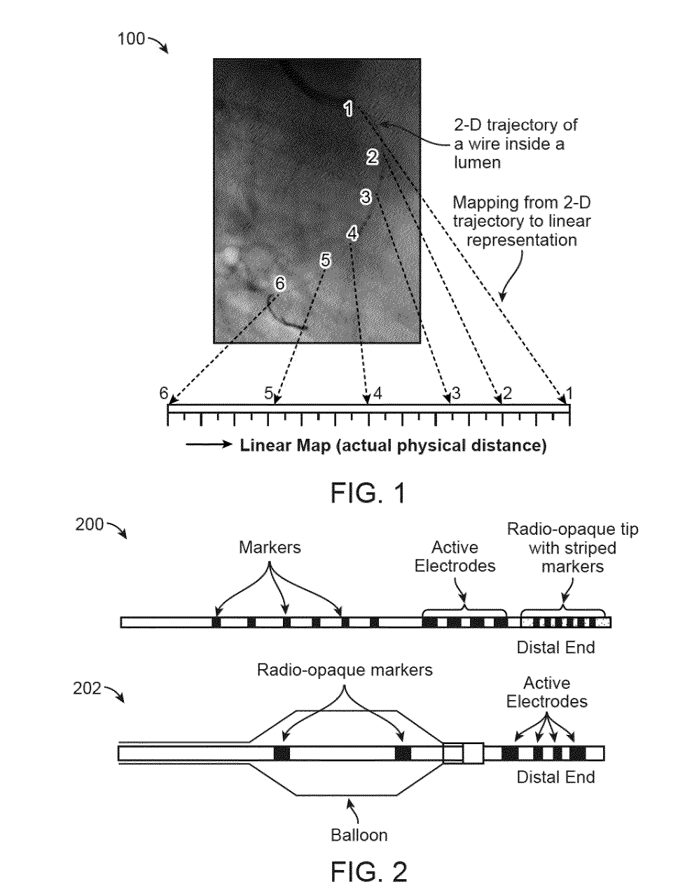

Here we describe methods to process the 2-D images to arrive at a linearized representation of a lumen of a moving organ. Illustrations of the proposed methods are shown for intervention of the coronary artery. A linear map is a mapping from a point on the curved trajectory of a lumen (or the wire inserted into the lumen) to actual linear distance measured from a reference point. This is shown in the schematic 100 in FIG. 1.

Many cardiac procedures involve the insertion of a lumen assessment device such as IVUS, OCT and LFR (see e.g., U.S. Pat. No. 8,374,689 B2 which is incorporated herein by reference in its entirety and for any purpose). In most of these cases, there is a need to co-register the position of the assessment device on a previously captured X-ray image that is usually also an angiographic image. This reference image is typically also used by the physician during a possible following intervention procedure such as angioplasty and stent deployment. A correct co-registration of the intervening device is important for optimal placement of stent. There is often another procedure following the deployment of stent where dilatation is performed. This procedure also requires an accurate registration of the device position in a reference frame. In all such cases, there is a need for having a common reference for the position of devices across these various steps. On this frame, the parameters and locations corresponding to all previous steps are super-imposed. Further, the location and parameters of the current step in the procedure would also need to accurately mapped and tracked in real time or near real time. To achieve this, there needs to be a method that can detect a device on a live X-ray and then map the detected position on to the reference image after compensating for motion due to heart beat, and breathing and other minor movements of the subject. Another need is to compensate for the foreshortening effect due the angle presented by the lumen trajectory to the viewing angle of the X-ray camera (e.g., motions of a C-arm relative to the patient) or motion of a platform upon which the patient is positioned (e.g., movement of a gurney or surgical table upon which the patient is placed). Such steps may be accomplished by one or more processors which are programmed accordingly.

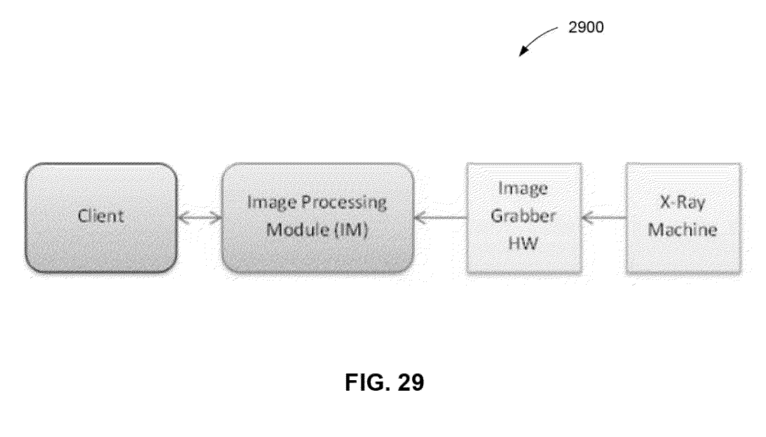

One method for accomplishing this for linear mapping of lumens is described in U.S. Pat. App. 61/644,329 filed May 8, 2012 and PCT/US2013/039995 filed May 7, 2013 (WO 2013/169814, designating the US), each of which is incorporated herein by reference in its entirety and for any purpose. An example of a typical system is illustrated in the block diagram 2900 shown in FIG. 29. The X-ray live-feed from an X-Ray Machine may be captured through an Image Grabber Hardware such as a capture card and sent to an image Processing Module (IM), which runs the algorithms for detection, tracking and co-registration.

Note that in some sections of the blood vessel, the actual lumen trajectory in 3-D may be curving into the image (i.e. it subtends an angle to the viewing place). In these cases, an apparently small section of the lumen in the 2-D curved trajectory may map to a large length in the linear map. The linear map represents the actual physical distance traversed along the longitudinal axis of the lumen when an object traverses through the lumen.

As shown in diagram 2900, the images from an X-Ray Machine are captured by an Image Grabber and sent to the IM. The IM runs the algorithms and interacts with a Client that uses the results of the IM. In some cases, there could be an interaction between the Client and IM to allow the Client to control the sequence of steps. The user may interact only with the Client in one variation.

When a session is ready to start, the user may be required to initiate the session through an interaction. This is typically done just prior to the angiogram taken before IVUS pullback, and which is recorded at the same angle with which IVUS pullback would take place. At this point, two possible options for user interaction may result. In the first option, the user provides a reference to the guide catheter (GC) tip from images provided by the IM. In the second option, the user does not have to provide a reference to the GC tip. The IM automatically detects the tip of the GC by automatically detecting the first angiogram that is performed after initiation. This automatic detection is done using several cues that are obtained from the sequence of image frames:
The X-ray opaque dye rapidly appears in the sequence of images. A comparison with previous images in the sequence clearly shows the presence of the dye. In particular, if a previous frame captured at the same or similar phase of heart beat is used for comparison, the difference stands out even more clearly.
The network of arteries that show up as dark tubular objects in the image frame. These have distinctive features that would not normally exist in a plain X-ray image. By selectively looking for these features, the presence of dye in the network of arteries can be detected in any single image frame

This angiogram is then analyzed to determine the position of the GC tip. One method is by identifying the end of main trunk of network of arteries from where the dye emanates. This think is then further analyzed especially in future frames when the dye fades away. The GC tip would still persist as an object visible under X-ray. The time evolution of spread of dye through the arteries is another cue used to identify the GC tip. The GC tip is then tracked automatically across all frames.

Alternately, GC tip can also be detected automatically without the use of an angiogram. The guide catheter has a distinct tubular structure. Image enhancement tailor made to enhance tube-like structures can be used to highlight similar structures. Any tube-like structures in the background of the image (for e.g. ribs) may also get highlighted in such a process. Analyzing all these highlighted structures for motion across different phases of the heart-beat can help separate the background structures from the object of interest (guide catheter).

The next step is to detect the tip section of the guidewire. This section is the most prominently visible feature visible in the image, and is detected with good robustness. Once the positions of the two-ends of the guidewire are reliably found, the intermediate section of the guidewire is detected and tracked. The algorithm used to detect the guidewire is inherently robust. Image processing algorithms selectively extract features that can discriminate guidewire shaped objects, thus allowing for effective detection. Further, there are other mechanisms built in to ensure robust detection of the entire guide wire even in difficult situations where the guidewire is not completely visible. These include narrowing down of segments of the frame to be analyzed by using the GC tip and the detected angiogram, using past fluoro images captured at the same phase of the heart cycle, applying appropriate models and physical constraints on the trajectory of the guidewire, and selectively looking for objects that are consistent with the periodic movement due to heartbeat.

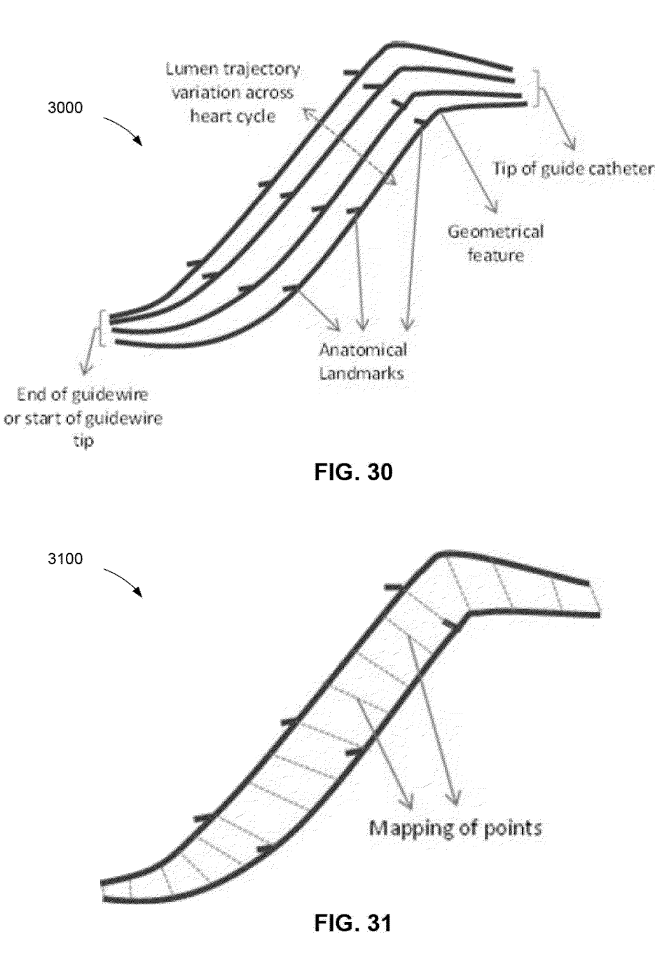

At the time of an angiogram, injection of dye is automatically detected when the artery gets lighted up. This detection triggers the algorithm pertaining to analysis of artery paths. Anatomical assessment is performed on the angiogram and distinct landmarks including branching points and lumen profile in the artery are identified across different phases of the heart-beat. These landmarks serve as anchor points around which a correspondence between points on the artery across phases of the heart are obtained. Further, the shape of the trajectory reveals properties such as curvature, points of inflexion which are preserved to a large extent across the heart cycle. The endpoints of the trajectory are also known—these are the tip of the guide catheter and the start of the tip section of the dense tip section of the guidewire. All of these are used to create an accurate point correspondence. The anatomical landmarks, distinctive geometrical features such as points of inflexion, curvature or other distinctive features (geometrical landmarks), and end points are the anchor points that are directly mapped to the corresponding point in the lumen trajectory for each phase of the heart cycle. Other points are interpolated around these anchor points while ensuring a smooth transition. The shape of the curve (e.g. flat sections are mapped to flat sections, and curved sections are mapped to curved sections) and distinctive lumen profile (e.g. focal lesions identified in one phase of the heart beat are mapped to its counterpart in other phases) are also taken into account. One example of these landmarks and features is illustrated in the diagram 3000 of FIG. 30 which shows lumen trajectory variations across the heart cycle.

The accuracy of mapping of points can be further enhanced in the case a device is inserted in a future step that helps in linearizing the lumen trajectory. This linearizing would map observed pixel distance which is affected by foreshortening effect to actual physical distance along the axis of the artery. In cases where the linearizing is not possible/performed based on the inserted device, the information regarding direction of motion of the device, speed of motion along the longitudinal direction of the lumen (if known) can further be used for refining the coregistration. For example, if the pullback is a known constant speed, this a priori information can be used to correct for small errors in co-registration by imposing appropriate constraints such as smoothness. Further, knowledge of foreshortening angle can be used for even tighter constraints.

An example of mapping of points on lumen trajectories across two phases of the heart cycle is shown in diagram 3100 of FIG. 31. Any pair of mapped points corresponds to the same physical location in the anatomy of the blood vessel.

Similarly a map is created all trajectories corresponding to neighboring phases of the heart cycle. The density of points to be mapped is determined by the need of the application.

From multiple angiographic images, the one that best illuminates the arteries and branches is selected and communicated to the client as a reference angiogram. Angiographic images corresponding to all phases of the heart cycle are stored internally in the IM for future reference.

In the next step, a lumen assessment device is inserted into the artery. This device typically is identifiable under X-ray and is detected and tracked across frames. Often there are one or more distinct marker-like features on the device that can be detected and tracked. Detection of the guidewire in a previous step significantly helps in reducing the search-space for IVUS/OCT marker detection. Any resultant translation because of the movement of C-arm or the patient table and changes in scale of the image is estimated and accounted for in tracking all the objects of interest. Tracking the locations of the markers of the device during insertion helps in estimating the foreshortening effect in different parts of the artery, thereby further enhancing the robustness of co-registration. As used herein throughout, “markers” may refer not only to radio-opaque markers or bands which are typically used on any number of endolumenal or elongate instruments, etc., for enhancing visibility under x-rays, x-ray fluoroscopy, etc., but may also refer to any x-ray observable feature (e.g., markers, bands, stents, etc.) in, on, or along the elongate instruments.

When the pullback of the lumen assessment device commences, the guidewire and the device that run over it are continually detected and tracked. Each image frame is mapped to one of the previously recorded set of reference angiographic frames. This mapping is done based on the phase of the heart cycle. This mapping can be done using the ECG corresponding to the same timestamp as the image timestamp, which is then used to identify the phase of the heart beat within that heart cycle. It can also be done comparing of the detected trajectory of the guidewire and device with the lumen trajectory of each of the recorded angiographic reference images. By correlating the lumen trajectory with each lumen trajectory corresponding to the set of reference angiographic frames, the one that matches best is selected as the matching phase. The point correspondence between that phase of the heartbeat and the phase that was provided to client is already known. This is used to map the position of the lumen assessment device on to the reference angiographic frame provided to client. This mapping is further refined based on the knowledge of the speed of pullback of the device (if it is uniform, and known), and using raw results from past and future frames. The estimated foreshortening during device insertion is an additional factor taken into account for refining the mapping. The final refined mapping is sent to the client as the co-registered location for the assessment device.

It should be noted here that only the angiogram needs to be recorded at a high enough frame rate to capture the variations during the heart cycle (e.g., 15 fps or 30 fps). This is consistent with current practice. However, when lumen assessment is performed, the recording could be done at a lower frame rate. For example it could be at 1 fps. Even though this low frame rate would often produce frames that are very different in phase compared to the reference angiogram, it is still possible to map the positions on to the reference angiogram using the point correspondence already established. This low frame rate allows reducing the amount of radiation that the patient is exposed to, which is very desirable. Alternatively, the low frame rate could be ECG gated, which allows capture at only a particular phase of the heart cycle. The reference angiogram is also recorded at the same phase, making it easy to register the location on the reference angiogram.

The previous section refers to a co-registering method mostly for a lumen assessment device that uses constant pullback. The same principles are also applicable if the pullback is not uniform. It is also applicable for a therapeutic procedure such as stent deployment.

Motion due to breathing is much less significant compared to motion due to the heart cycle. This has been observed during multiple animal experiments. It can be considered to be composed of following components; a) Global translation b) Global rotation around the axis perpendicular to the plane of viewing c) Global rotation around an axis that is in the plane of view and d) distortions in the trajectory of the vessel. Of these, Global rotation around an axis that is in the plane of view and distortions in the trajectory of the vessel give insignificant residual errors in co-registration and are only partially addressed by our algorithm. The algorithm fully accounts for Global translation and the global rotation around the axis perpendicular to the viewing plane—these are affine transformations that are estimated and corrected for.

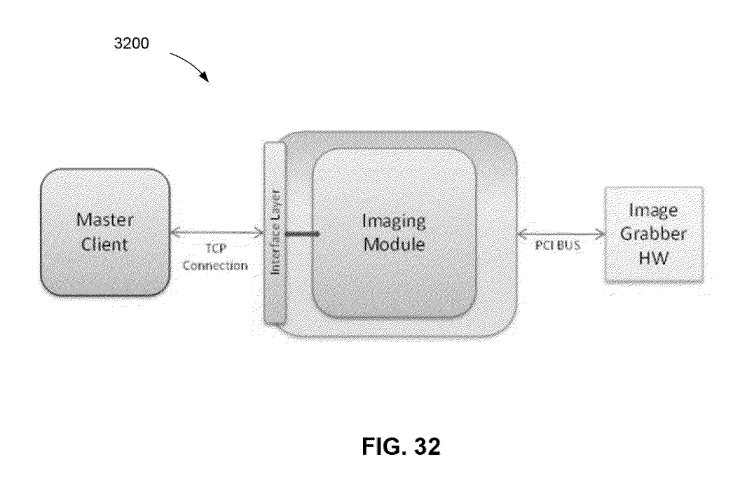

As shown in the FIG. 32, one particular implementation for utilizing the methods described above is shown in the diagram 3200 of FIG. 32. In this example, an X-ray live-feed is captured through, e.g., an Image Grabber HW such as a capture card, and is sent to the Imaging Module which runs the co-registration algorithm, as previously described. In this instance, the guide catheter and the guidewire may have been already placed.

The linear mapping and co-registration methods are applicable in a procedure using any one of the following endo-lumen instruments in a traditional (2-D) coronary angiography:
1. Guidewire with active electrodes that are radio-opaque and/or markers as disclosed in herein above.
2. Catheter with electrodes that are radio-opaque as disclosed hereinabove used with a standard guidewire
3. A standard guidewire used with a standard angioplasty, pre-dilatation or stent delivery catheter containing radiopaque markers.
4. Any catheter (IVUS, OCT, EP catheters), guidewire, or other endo lumen devices that have at least one radiopaque element (that can be identified in the X-ray image).

Apart from the above mentioned devices, a similar approach can also be used for obtaining a linear map in coronary computed tomography (3-D) angiographic images and bi-plane angiographic images, using only a standard guidewire. The linear map generation can later be used for guiding further cardiac intervention in real-time during treatment planning, stenting as well as pre- and post-dilatation. It can also be used for co-registration of lumen cross-sectional area measurement measured either with the help of QCA or using multi-frequency electrical excitation or by any other imaging (IVUS, OCT, NIR) or lumen parameter measurement device where the parameters need to be co-registered with the X-ray. Standard guidewire and catheter as well as guidewire and catheter with added electrodes and/or markers are referred to as an endo-lumen device in the rest of the document. Also as previously mentioned, markers may refer to any x-ray observable feature (e.g., markers, bands, stents, etc.) in, on, or along the endo-lumen device or any elongate instruments.

Construction of Guidewire and Catheter with Markers and/or Electrodes

FIG. 2 illustrates the construction of a guidewire 200 and catheter 202 with active electrodes and markers as shown. The spacing and sizes are not necessarily uniform. The markers and electrodes are optional components. For example, in some embodiments, only the active electrodes may be included. In other embodiments, only the markers or a subset of markers may be included. If the guidewire 200 has no active electrodes or markers, it is similar to a standard guidewire. Even without the markers or electrodes, the guidewire is still visible in an X-ray image. The coil strip at the distal end of a standard guidewire is made of a material which makes it even more clearly visible in an X-ray image. If the catheter 202 does not have active electrodes, it is similar to a standard balloon catheter, which has a couple of radio-opaque markers (or passive electrodes) inside the balloon.

There are several modifications and variations possible to the illustrated constructions in terms of geometry, locations, number and size of markers/electrodes as well as spacing between them. Apart from using active electrodes for linearizing, the guidewire 200 and catheter 202 may be constructed with multiple radiopaque markers which are not necessarily electrodes. Radiopaque markers in a guidewire are shown in FIG. 2. It can either be placed on the proximal side or distal side of the active electrodes. It can also be placed on both the sides of the active electrodes or could be replace them for the purposes of artery path linearization. If the markers on the proximal side of the electrodes span the entire region from the location of the guide-catheter tip to the point where the guidewire is deep-seated, linearization can be done independently for each phase of the quasi-periodic motion. But such constructions are often not desired during an intervention as it often visually interferes with other devices or regions of interest. Hence a reduced set of markers are often desirable. Apart from these, another configuration of the possible guidewire would be to make the distal coil section of the guidewire striped with alternating strips which are radiopaque and non-radiopaque in nature, of precise lengths which need not necessarily be uniform. These proposed modifications may be used independently or together in any combination for artery path linearization. The distal radiopaque coil section of a standard guidewire (without it being striped) can also be used for getting an approximate estimate of the linearized map of the artery. This estimate becomes more and more accurate as the frame rate of the input video increases. All of these variations are anticipated and within the scope of this invention.

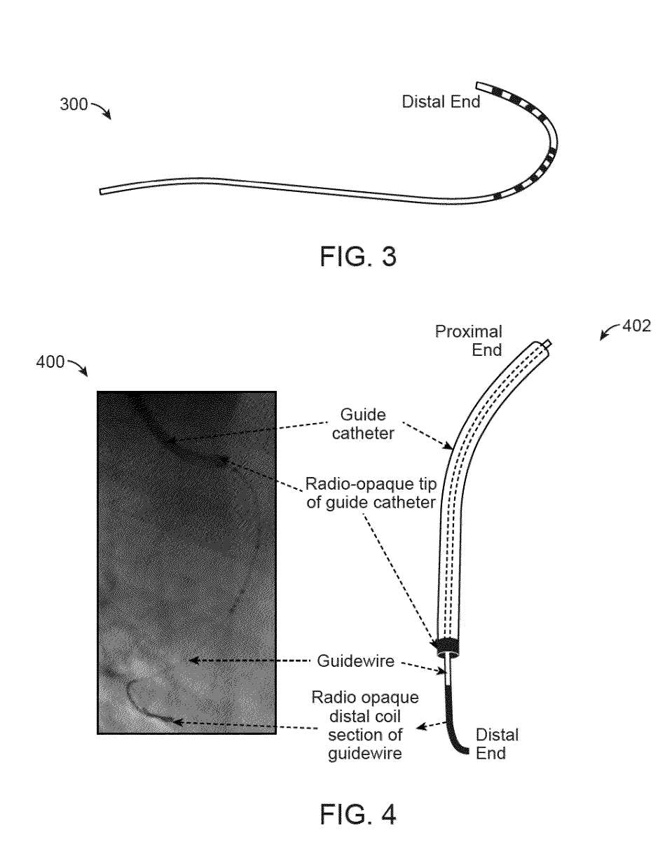

When the endo-lumen device is inserted into an artery, it follows the contours of the artery. When a 2-D snapshot of the wire is taken in this situation, there would be changes in the spacing, sizes and shapes of the electrodes depending on the viewing perspective. For instance, if the wire is bending away from the viewer, the spacing between markers would appear to have reduced. This is depicted by the curved wire 300 shown in FIG. 3.

Description of Various Use Cases

This sub-section describes the various use cases in which the generation of a linearized map would be of clinical significance.

When the linearization is done using guidewire with markers and electrodes, or using standard guidewire, the linearized map can be used for co-registration of anatomic landmarks (such as lesions, branches etc.) with lumen measurement.

Such co-registration can serve several purposes:
1. The points of interest in such as a lesion can be then superimposed back onto an angiographic view
2. Other therapy devices (such as stent catheters, balloon catheters) can be guided to the region of interest
3. Alternatively, the advancement of any device along the co-registered artery can be displayed in the linear view to guide therapy.

If a standard-guide wire is used along with a catheter consisting of markers/electrodes, and the markers or electrodes in catheter is used for linearization during pre-dilatation, computer-aided intervention assistance can be provided for all the further interventions. This holds well even if the linearized map is generated using standard catheter containing radiopaque balloon markers. Once linearized, the artery map which is specific to the patient can also be used for other future interventions for the patient in that artery.

Obtaining Live Video Output, ECG and Other Vital Signs from the Medical Imaging Device

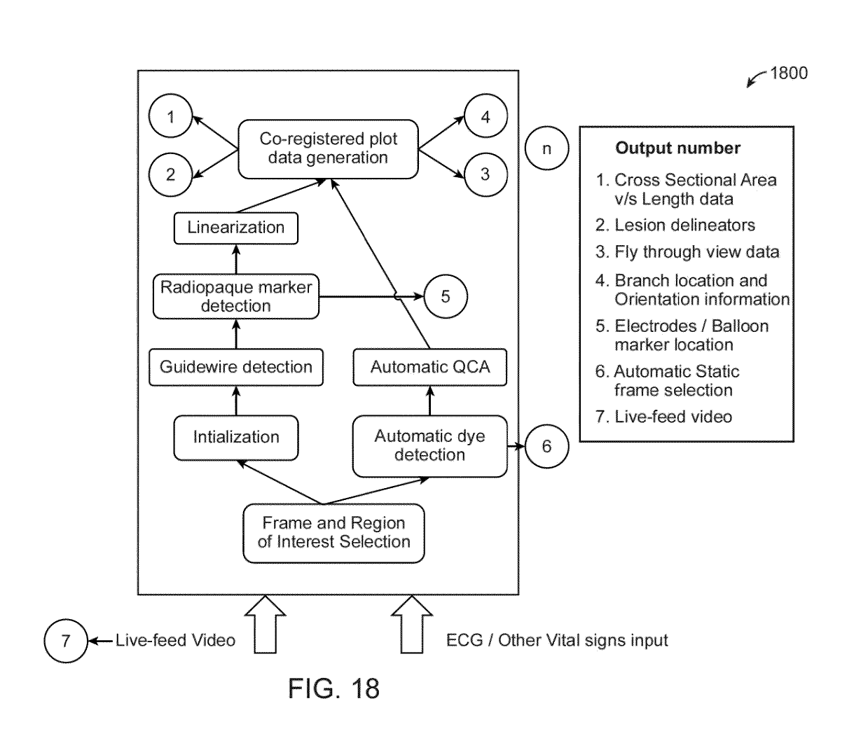

FIG. 18 presents a block diagram 1800 of the details of various modules of the invention along with the output provided to the end user. Each of the various modules is described in further detail herein. DICOM (Digital Imaging and Communications in Medicine) is a standard for handling, storing, and transmitting information in medical imaging. But, it is generally available for offline processing. For the system that is proposed in this invention, live video data, as seen on a display device which an interventionalist uses, is required. For this purpose, either the output of the medical imaging device or the signal that comes to the display device is duplicated. The video input to the display device can either be digital or analog. It can be in interlaced composite video format such as NTSC, PAL, progressive composite video, one of the several variations/resolutions supported by VGA (such as VGA, Super VGA, WUXGA, WQXGA, QXGA), DVI, interlaced or progressive component video etc. or it can be a proprietary one. If the video format is a standard one, it can be sent through a wide variety of connectors such as BNC, RCA, VGA, DVI, s-video etc. In such a case, a video splitter is connected to the connector. One output of the splitter is connected to the display device as before whereas the other output is used for further processing. In cases where the video out is in proprietary format, a dedicated external camera is set up to capture the output of the display device and output of which is sent using one of the aforementioned type of connectors. Frame-grabber hardware is then used to capture the output of either the camera or the second output of video splitter as a series of images. Frame grabber captures the video input, digitizes it (if required) and sends the digital version of the data to a computer through one of the ports available on it such as—USB, Ethernet, serial port etc.

Time interval between two successive frames during image capture (and thus the frame rate of the video) using a medical imaging device need not necessarily be the same as the one that is sent for display. For e.g. some of the C-arm machines used in catheter labs for cardiac intervention has the capability of acquiring images at 15 and 30 frames per second, but the frame rate of the video available at the VGA output can be as high as 75 Hz. In such a case, it is not only unnecessary but also inefficient to send all the frames to a computer for further processing. Duplicate frame detection can be done either on the analog video signal (if available) or a digitized signal.

For duplicate frame detection in the analog domain, comparing the previous frame with the current frame can be done using a delay line. An analog delay line is a network of electrical components connected in series, where each individual element creates a time difference or phase change between its input signal and its output signal. The delay line has to be designed in such a way that it has close to unity gain in frequency band of interest and has a group delay equal to that of duration of a single frame. Once the analog signal is passed through the delay line, it can be compared with the present frame using a comparator. A comparator is a device that compares two signals and switches its output to indicate which is larger. The bipolar output of the comparator can either be sent through a squarer circuit or through a rectifier (to convert it to a unipolar signal) before sending it to an accumulator such as a tank circuit. The tank circuit accumulates the difference output. If the difference between the frames is less than a threshold, it can be marked as a duplicate frame and discarded. If not, it can be digitized and sent to the computer.

In our implementation we have used a digital duplicate frame detector. Previous frame is compared with the present frame by computing sum of squared differences (SSD) between the two frames. Alternately sum of absolute differences (SAD) may also be used. Selection of threshold for selection and rejection of frames has to be adaptive as well. Threshold may be different for different x-ray machines. It may even be different for the same x-ray machines at different points of time. Selecting and rejecting the frames based on a threshold is a 2 class classification problem. Any 2 class classifier may be used for this purpose. In our implementation, we chose to exploit the observation that the histogram of SSD or SAD is typically a bimodal histogram. One mode corresponds to the set of original frames. The other mode corresponds to the set of duplicate frames. The selected threshold minimized the ratio of intra-class variance to inter-class variance.

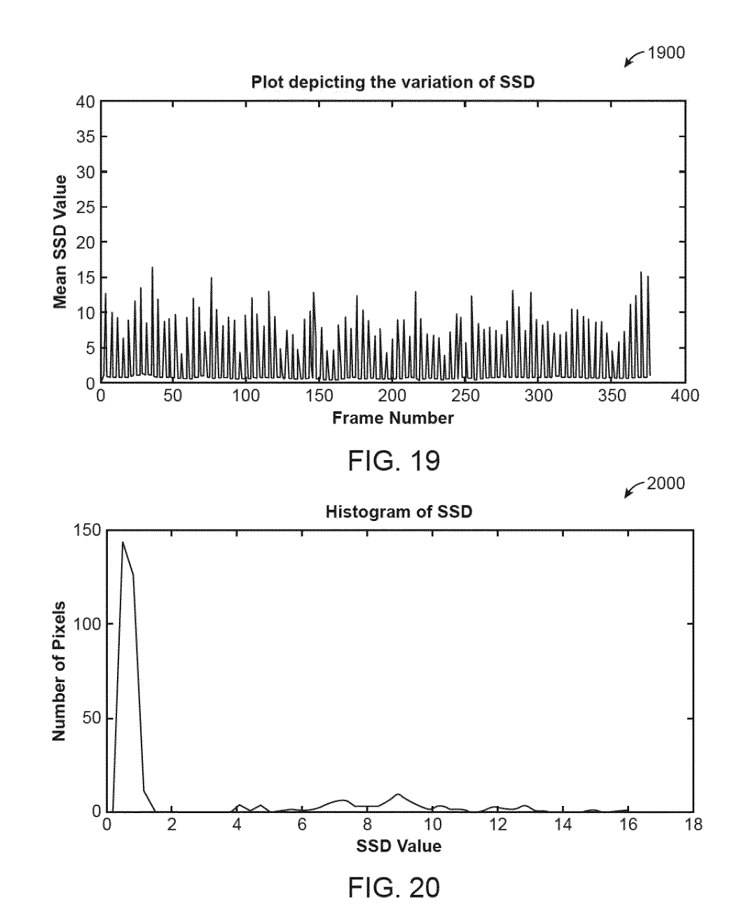

For experimentation purpose, a video with 15 frames per second was displayed at 60 frames per second. FIG. 19 shows a plot 1900 of the variation of mean SSD value computed after digitizing the analog video output of the display device. It can be noted from FIG. 19 that the SSD value has local maxima once in every 4 frames. FIG. 20 illustrates a bimodal histogram 2000 of SSD with a clear gap between the 2 modes.

In our implementation of the proposed system, the video after duplicate frame detection is sent as output from the hardware capture box. This is output number 7 as seen in FIG. 18.

Vital signs on the other hand are easier to tap. ECG out for example typically comes out from a phono-jack connector. This signal is then converted to digital format using an appropriate analog to digital converter and is sent to the processing system.

Automatic Frame and Region of Interest Selection

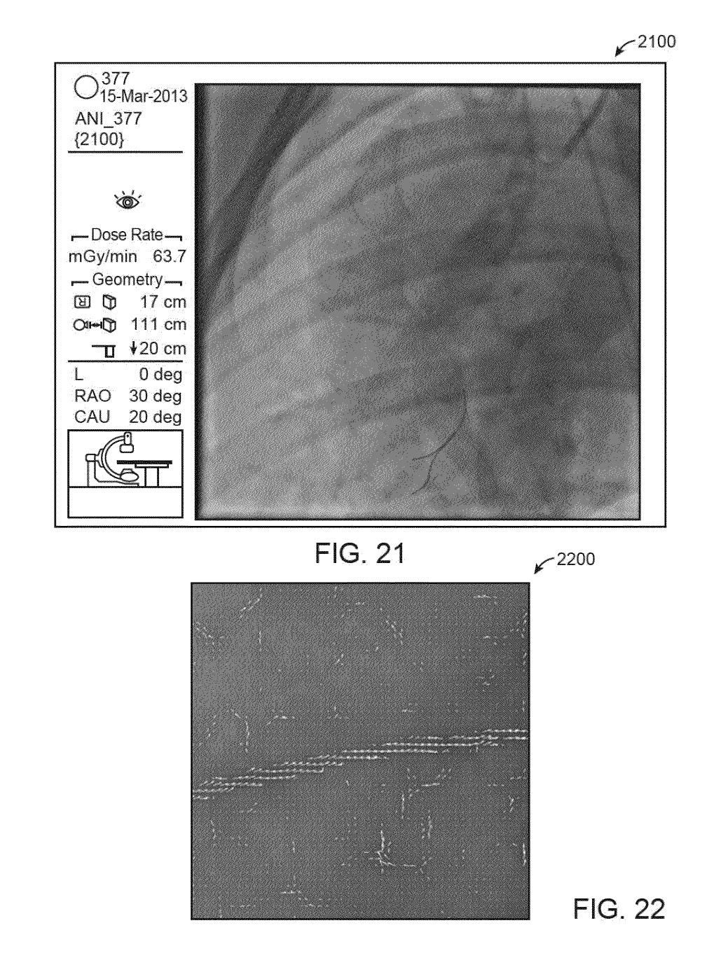

While processing a live feed of images, not all the frames are useful. An effective data selection algorithm lets you select the images and regions of interest automatically. Unlike DICOM image, live feed data often have several tags embedded on it. For example, FIG. 21 shows a typical live-feed data 2100 captured from a cardiac intervention catherization lab. An intensity based region of interest selection is used to select appropriate region for further processing.

Similarly, during an intervention, the medical imaging device need not necessarily be on at all points of time. In fact, during cardiac intervention using C-arm X-ray machine, radiation is switched on only intermittently. In such a case, the output at the live feed connector is either a blank image, or an extremely noisy image. Automatic frame selection algorithm enables the software to automatically switch between processing the incoming frames for further analysis or dump the frames without any processing.

Tracking of endo lumen device covers initialization, guidewire detection and radiopaque marker detection as mentioned in FIG. 18 and as also disclosed in a number of co-owned patents and patent applications incorporated hereinabove.

Description of the Algorithm

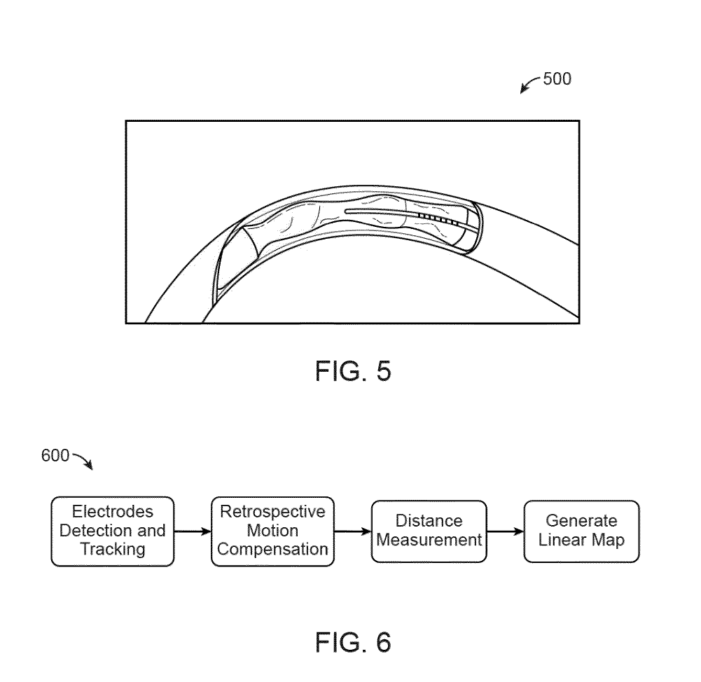

The guidewire, guide catheter and catheter used in an angiographic procedure are shown in the fluoroscopic image 400 of FIG. 4. The guidewire and guide catheter 400 are further shown illustrating how the guidewire may be advanced from the catheter. An illustration of the radiopaque markers 500 on a guidewire inside a coronary artery is shown in FIG. 5.

The algorithm that is described here is for linearization of a lumen with reduced set of markers. Markers spanning the entire length of the artery can be seen as a special case of this scenario. For achieving the goal of artery path linearization, we detect the radiopaque markers (either active electrodes in the guidewire and catheters or balloon markers) in the endo-lumen device and track them across frames through different phases of heart-beat. Retrospective motion compensation algorithm is then used to eliminate the effect of heart-beat and breathing for measuring the distances travelled by the electrodes within the artery. The measured distance in pixels is converted to physical distance (e.g. mm) in order to generate a linearized map of the geometry of the coronary artery. FIG. 6 shows a block diagram 600 of an overview of the steps involved.

The challenges in achieving each of these tasks are described below. The radiopaque nature of the markers makes them quite prominently visible in an angiographic image. Several methods such as edge-detectors, interest-point detectors, template matching, Hough-transform based methods may be used for detecting the electrodes individually. However, maintaining robustness in the presence of other radiopaque objects such as pacemaker leads and coronary artery bypass graft wires etc. is a challenging task.

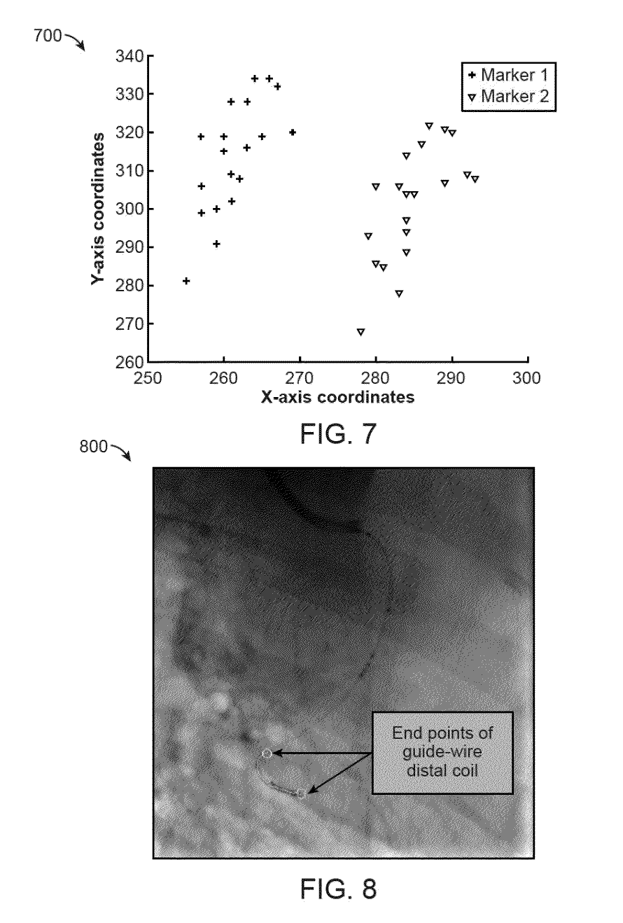

Due to motion observed in an imaged frame, the coordinates of the electrodes in an image need not necessarily remain constant even if the endo-lumen device is kept stationary. It should be noted that the observed motion in an imaged frame could be a result of one or more of the following occurring simultaneously: translation, zoom or rotational changes in the imaging device; motion due to heart-beat and breathing; physical motion of the subject or the table on which the subject is positioned. FIG. 7 illustrates a chart 700 showing the changes in position of two markers in different phases of the heart-beat when the guidewire is stationary.

To compensate for motion of the electrodes, a retrospective motion correction or motion prediction strategy may be used. However, image-based motion correction algorithms are usually computationally expensive and may not be suitable for real-time applications. In our implementation, we segment the image for identifying guidewire. In one embodiment, the entire guidewire is used for motion correction while in another embodiment only a portion of the guidewire in the region of interest is used for motion correction.

In this process, the guidewire is detected in every frame in a manner described later in this section. Markers and electrodes, if any, are also detected in this process. Once the guidewire is robustly detected, known reference points on the guidewire system (guidewire and any catheter it may carry) are matched between adjacent image frames, thereby determining and correcting for motion due to heartbeat between the frames. These reference points may be end points on the guidewire, the tip of the guide catheter, or the distal radio-opaque section of the guidewire, or any marker that has not moved significantly longitudinally due to a manual insertion or retraction of the endo-lumen instrument or any anatomical landmark such as branches in an artery. When the guidewire markers are used for linearization, these markers by definition are not stationary along the longitudinal lumen direction and hence should not be used as land mark points.

Since the trajectory of the catheter is equivalent to that of the guidewire, motion compensation applicable to the guidewire is equally applicable to the catheter. Note that the catheter may actually be moving over the guidewire due to a manual insertion or retraction procedure. Hence the catheter markers should not be used for motion compensation when the catheter is not stationary. In fact, after motion compensation, the movement of the markers on the non-stationary endo lumen device is tracked to determine the position of the device within the lumen.

The segmentation of the guidewire in one frame enables one to narrow down the search region in a subsequent frame. This allows for reduction in search space for localizing the markers as well as making the localization robust in the presence of foreign objects such as pacemaker leads. However, detection of the entire guidewire in itself is a challenging task and the markers are usually the most prominent structures in the guidewire. Hence, our approach considers detecting electrodes and segmenting the guidewire as two-interleaved process. The markers and the guidewire are detected jointly, or iteratively improving the accuracy of the detection and identification, with each iteration, until no further improvement may be achieved.

Motion compensation achieved through guidewire estimation can be used for reducing the amount of computation and the taking into account the real-time need of such an application. However, as mentioned earlier in the section, image-based motion compensation or motion prediction strategy may be used to achieve the same goal by using a dedicated high-speed computation device. The resultant motion compensated data (locations of endo-lumen devices in case of guidewire based motion compensation; image(s) in case of image-based motion compensation) can be used to compute translation of endo-lumen devices/markers along the longitudinal axis of a lumen. This computed information can further be visually presented to the interventionalist as an animation or as series of motion compensated imaged frames with or without endo-lumen devices explicitly marked on it. The location information of the markers and other endo-lumen devices can also be superimposed on a stationary image.

Algorithms for guidewire segmentation as well as algorithms for electrode detection across all the frames are further described in detail herein. Moreover, algorithms for motion compensation through finding the point correspondences between the guidewires in adjacent frames are discussed followed by linear map generation.

Guide Wire Segmentation and Electrode Localization

In one method, our approach for guidewire segmentation comprises four main parts:
1. Reliable detection of the end points of the guidewire.
2. Enhancement of tubular objects in the image.
3. Detection of an optimum path between the two end-points where the optimality is based on continuity of the curve as well as its traversal through the tube-like structures.
4. Localization of the markers in the vicinity of the guidewire and re-estimation of the guidewire segmentation based on the detected markers.

Detection of the End-Points of the Guidewire

Detection of the end-points of the guidewire comprises detecting known substantial objects in the image such as the guide-catheter tip and the radiopaque guidewire strip. These reference objects define the end points of the guidewire. An object localization algorithm (OLA) that is based on pattern matching is used to identify the location of such objects in a frame. In one embodiment of the invention, a user intervenes by manually identifying the tip of the guide catheter by clicking on the image at a location which is on or in the neighborhood of the tip of the guide catheter. This is done in order to train the OLA to detect a particular 2-D projection of the guide-catheter tip. In other embodiments, the tip of the guide catheter is detected without manual intervention. Here, the OLA is programmed to look for shapes similar to the tip of the guide catheter. The OLA can either be trained using samples of the tip of the guide catheter, or the shape parameters could be programmed into the algorithm as parameters. In yet another embodiment, the tip of the guide catheter is detected by analysing the sequence of images as the guide catheter is brought into place. The guide catheter would be the most significant part that is moving in a longitudinal direction through a lumen in the sequence of images. It also has a distinct structure that is easily detected in an image. The moving guide catheter is identified, and the radio opaque tip of the guide catheter is identified as the leading end of the catheter. In yet another embodiment, tip of the guide catheter is detected when the electrodes used in lumen frequency measurement as previously described move out from the guide catheter to blood vessel. The change in impedance measured by the electrodes change drastically and this aids in guide catheter detection. It can also be detected based on injection of dye during an intervention.

The radio-opaque tip of the guide catheter represents a location that marks one end of the guidewire. The tip of the guide catheter needs to be detected in every image frame. Due to the observed motion in the image due to heart-beat, location of the corresponding position in different frames varies significantly. Intensity correlation based template matching approach is used to detect the structure which is most similar to the trained guide-catheter tip, in the subsequent frames. The procedure for detecting can also be automated by training an object localization algorithm to localize various 2-D projections of the guide-catheter tip. Both automated and user-interaction based detection can be trained to detect the guide-catheter even when the angle of acquisition through a C-arm machine is changed or the zoom factor (height of the C-arm from the table) is changed. It is assumed throughout the process of linearization that guide catheter tip is physically unmoved. This assumption is periodically verified by computing the distance of the guide catheter tip with all the anatomical landmarks, such as the location of the branches in the blood vessel. When the change is significant even after accounting for motion due to heart-beat, the distance moved is estimated and compensated for in further processing. Locating the branches in the blood vessel of interest is described further herein.

Tip of the guidewire being radiopaque is segmented based on its gray-level values. The radio-opaque tip of the guide catheter represents a location that represents one end of the guidewire section that may be identified.

Once the tip of the guide catheter is identified, the next step is to identify the radiopaque coil strip of the guidewire, which represents the other end of the guidewire that needs to be identified. In some situations, the guide catheter is detected before the guidewire is inserted through the distal end of the guide catheter. In such situations, the radiopaque coil strip at the distal end of the guidewire is detected automatically as it exits out of the guide catheter tip by continuously analyzing a window around the guide catheter tip in every frame. In other situations (other embodiment), the distal radiopaque coil strip of the guidewire is identified by user intervention. The user would be required to select (e.g. though a mouse click) a point that is in the vicinity of the proximal end (the end that is connected to the core of the guidewire) of the guidewire's coil strip. In yet another embodiment, distal end of the guidewire is detected based on its distinctly visible tubular structure and low gray-level intensity.

Since the radiopaque coil strip of the guidewire is strongly visible on an X-ray, it is relatively easy to detect the radiopaque distal end. A gray-level histogram of the image is created. A threshold is automatically selected based on the constructed histogram. Pixels having a value below the selected threshold are marked as potential coil-strip region. The marked pixels are then analysed with respect to connectivity between one another. Islands (a completely connected region) of marked pixels represent potential segmentation results for guidewire coil section. Each of the islands has a characteristic shape (based on the connectivity of the constituting pixels). The potential segmentation regions are reduced by eliminating several regions based on various shape-based criteria such as area, eccentricity, perimeter etc. of the inherent shapes and the list of potential segmentation region is updated. The region which has the highest tube-likeliness metric is selected as the guidewire coil section. Once the coil section is identified, starting from any arbitrary point on the coil section, a search in all the directions is performed to detect the two end points of the coil-strip. The end-point which is closest to that of the corresponding point in the previous frame or from that of the guide catheter tip is selected. This represents the second end point of the guidewire that needs to be identified for guidewire segmentation. The result of detection of the distal coil is shown in the image 800 of FIG. 8. There are 2 end points detected. Of these, the one closer to the point selected by the user is selected in the first image frame.

Due to the observed motion in the image due to heart-beat, location of the corresponding position in different frames varies significantly. Thus the location of guide-catheter tip and the proximal end of the guidewire coil strip changes significantly from frame to frame. To detect the end-points of the guidewire in all the subsequent frames, a region around the detected points in the initial frame is selected. The gray level intensities of the selected regions are considered as a template. A 2-D correlation is performed in a relatively large region around the detected coordinates in the subsequent frames. The location where the correlation score achieves a maximum is selected as the end-points of the guidewire in the sub-sequent frames. In cases where the global maximum is not ‘significant’ enough, several candidate points are selected. Motion between the previous frame and all the candidate points, distance of the guide catheter point in the current frame to the frame in the same phase, but several previous heart beats is computed. Resultant optimum point minimizes a combination of these distance functions. The algorithm for segmentation of guidewire uses the detected end-points as an initial estimate for rejecting tubular artifacts which structurally resembles a guidewire. Guidewire segmentation procedure also refines the estimate of the position of the end-points.

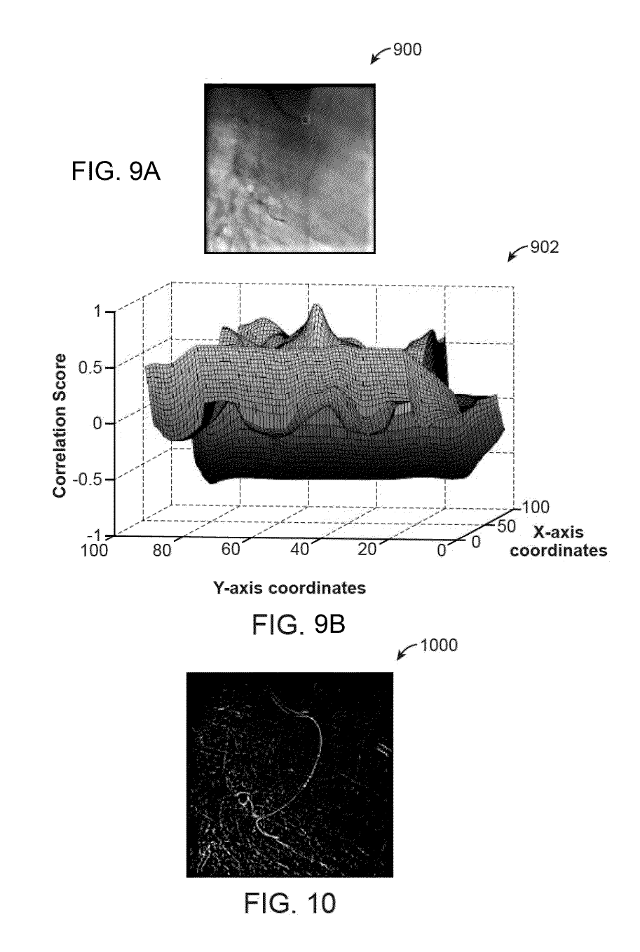

The result of detection of the tip of the guide catheter is shown in FIG. 9A which depicts a localized guide-catheter 900 having a tip based on template matching at one end of the guidewire and a marked tip of the guidewire radiopaque coil at the other end. FIG. 9B shows a chart 902 graphing the variation of the correlation score and the presence of a unique global maximum which is used for localization of the tip of the guide catheter.

The location of the end-points of the guidewire 900 change significantly when the angle of acquisition through a C-arm machine is changed or the zoom factor (height of the C-arm from the table) is changed. In such situations, the end-point information from the previous frame cannot be used for re-estimation. Either the user may be asked to point at the corresponding locations again or the automatic algorithm designed to detect the end-points without requiring an input from previous frame, as discussed earlier in the section, may be used. The detection of angle change of the C-arm can be done based on any scene-change detection algorithm such as correlation based detection. This is done by measuring the correlation of the present frame with respect to the previous frame. When the correlation goes lesser than a threshold, we can say that the image is considerably different which is caused in turn by angle change. Angle change can also be detected by tracking the angle information available in one of the corners of the live feed images captured (as seen in FIG. 21).

Enhancement of Objects of Interest

Several approaches can be found in the literature for enhancing specific objects of interest. In one embodiment where the objects of interest resemble tube-like structures image enhancement techniques specific to highlighting tube-like structures are used. Some of the commonly used metrics are Frangi's vesselness metric and tube-detection filter. In another embodiment, where the interventional tools do not resemble tube-like structures, image enhancement techniques specific to the geometry of the object of interest is used. To demonstrate implementation, we use Frangi's vesselness measure to enhance the tubular objects in the image. However, any alternative method which serves a similar purpose can be used as its substitute. In Frangi's formulation of tube-likeliness T(x) is defined as:

$$T ( x ) = \{ \begin{matrix}0 & if {λ}_{2} > 0 \\ ( 1 - exp ( - \frac{{s}^{2}}{2 {γ}^{2}} ) ) & otherwise\end{matrix} \} \qquad ( 1 )$$

where λ₁ and λ₂ are eigenvalues of the Hessian matrix of the image under consideration such that |λ₁|≦|λ₂| with S=\sqrt{λ₁²+λ₂²}.

The Hessian matrix is the second order derivative matrix of the image. For each pixel P(x,y) in the image there are four 2^{nd} order derivatives as defined by the 2×2 matrix

$$H ( x , y ) = [ \begin{matrix}\frac{{\partial }^{2} P}{\partial {x}^{2}} & \frac{{\partial }^{2} P}{\partial x \partial y} \\ \frac{{\partial }^{2} P}{\partial y \partial x} & \frac{{\partial }^{2} P}{\partial {y}^{2}}\end{matrix} ] .$$

The values α and β are weightage factors and are chosen empirically to yield optimal results.

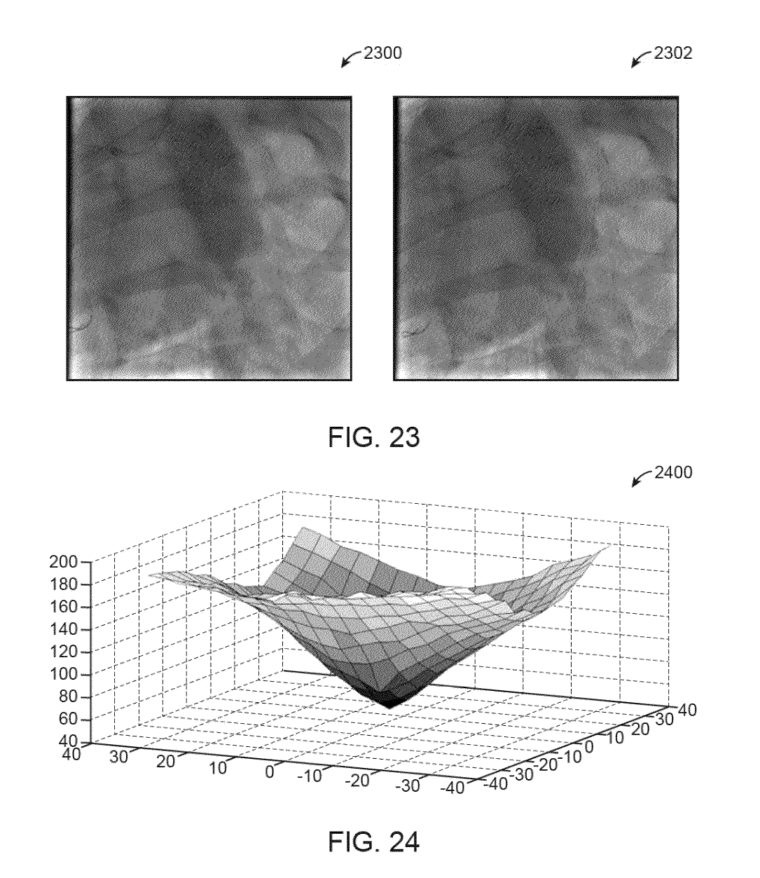

The result of enhancement 1000 of tubular objects is shown in FIG. 10 (whiter values correspond to pixels that are more likely to be part of a tubular structure; darker values denote lower likelihood). The tube-likeliness metric thus obtained is a directionless metric. For detecting the path of the endo-lumen device, dominant direction of the tube-likeliness metric is sometimes valuable information. For getting the dominant direction information, eigenvector of the Hessian matrix is used. FIG. 22 shows the directional tube-likeliness metric 2200 overlaid on an original image representative of the eigenvector overlaid on the image pixels.

Optimum Path Detection

In cases where linear translation of the C-arm position takes place or the zoom factor (height of the C-arm from the table) changes, motion of the same can be estimated. This estimation is based on analyzing the previous frame with respect to the current frame and computing a metric such as sum of squared differences (SSD) or sum of absolute differences (SAD) between the pixels of the images. SSD or SAD is computed for several possible combinations of translation and zoom changes between consecutive frames and the one with minimum SSD/SAD is selected as the correct solution of translation and zoom. For example, FIG. 23 shows 2 consecutive frames 2300, 2302 with slight translation (and no zoom factor change) between the 2 frames 2300, 2302. The SSD values are computed for a wide variety of possible translations varying from −40 to +40 pixels in both the directions. FIG. 24 illustrates a graph 2400 illustrating the variation of SSD values for different possible translations. Minimum is obtained for a translation of 4 pixels in one direction (X-axis) and 12 pixels along the other direction (Y-axis).

C-arm angle changes by a small amount can sometimes be approximated by a combination of translation and zoom changes. Because of this, it becomes essential to differentiate between rotation from translation and zoom changes. While processing a live-feed of images, translation is usually seen seamlessly whereas rotation by an angle, however small that is, causes the live-feed to ‘freeze’ for some time until the destination angle is reached. Effectively, live-feed video contains transition states of translation as well whereas during rotation, only the initial and final viewing angles are seen. In rare cases where the transition state is available in rotation as well, detection of the angle of C-arm as seen in live-feed video 2100 (lower left corner in FIG. 21) can be used to make this differentiation.

Guidewire Detection

Once the end-points of the guidewire are known as well as the tube-likeliness is computed for each pixel in the image, delineation of guidewire reduces to a graph-theoretic shortest-path problem with non-negative weights. More specifically, considering that image pixel in the image is a node, an edge connecting 2 pixels is a vertex, and weight of each vertex is inversely proportional to the tube-likeliness at that point, the guidewire segmentation algorithm can be rephrased as finding a path with the least path-distance. Since the weights under consideration are non-negative, Dijkstra's algorithm or live-wire segmentation which is very well known in the field of computer vision may be used for this purpose.

Alternatively, the segmentation problem may also be viewed as edge-linking between the guidewire end points using the partially-detected guidewire edges and tube-likeliness. Active shape models, active contours or gradient vector flow may also be used for obtaining a similar output.

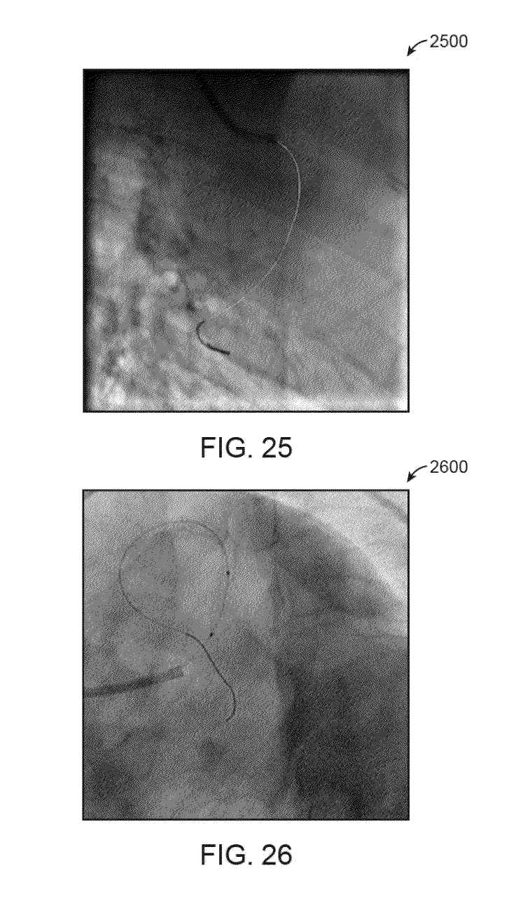

In our implementation, we use a modified version of Dijkstra's algorithm for segmenting and tracking the guidewire. The Dijkstra's algorithm implemented takes care of the smoothness of the curve being detected by giving some weighting to the previous pixels in the path from the starting point to the pixel under consideration. The search for optimum path is stopped when both the end points (as detected in initialization step) are processed. FIG. 25 highlights the detected guidewire 2500 by such an algorithm. This algorithm can also be used for tracking multiple endo-lumen devices inserted in different blood vessels simultaneously. Alternately, a regular Dijkstra's algorithm can be used to detect and track the guidewires and after they are detected, a separate smoothing function can be applied to obtain a smooth guide-wire.

In several practical scenarios, the guide-catheter tip and the guide-wire tip may go in and out of the frame due to heart beat. In such a case (where at least one of the end point is visible), modified Dijkstra's algorithm is started from one of the end point. Since one of the end-points is out of the frame, the pseudo end point for the optimum path detection algorithm is one of the border pixels in the image. The search for the optimum path is continued until all the border pixels in the image are processed. The path which is nearest to the previously detected guidewire (in the same phase of the heart beat) is chosen as the optimum path.

There is also a possibility of a section of the guidewire going outside a frame while both the end points are visible. In such a case, modified Dijkstra's algorithm is started from both the end-points assuming that only one end-point is visible (based on the above mentioned strategy). Results of both the end-points are combined and the partially absent guidewire path can be reconstructed assuming the continuity of the guidewire doesn't change in the absent region.

In yet another case where the 2-D projection of the 3-D path of the guidewire forms a self-loop, modified Dijkstra's algorithm is used to detect the path where no loop exists. The point where the change of path of the guidewire is abrupt, a separate region based segmentation technique is used to detect the loop in the guidewire. For example, in our implementation, fast marching based level set algorithm is used to detect the loop in the guidewire. This part of the algorithm is set off only in cases where there is a visible sudden change of guidewire direction. FIG. 26 shows an example of such a use case scenario where a self-loop 2600 is shown formed in the guidewire.

The search space of the Dijkstra's algorithm is also restricted based on the nearness of a pixel to the guidewire that was detected in the same phase in several previous heart-beats. The phase of the heart-beat can be obtained by analyzing the ECG or other measuring parameters that are coordinated with the heart beat such as pressure, blood flow, Fractional flow reserve, a measure of response to electrical stimulation such as bio-impedance, etc., obtained from the patient.

In our implementation, we have used ECG based detection of phase of the heart-beat. This is done by detecting significant structures in ECG such as onset and end of P-wave and T-wave, maxima of P-wave and T-wave, equal intervals in PQ segment and ST segment, maxima and minima in QRS complex. If a frame being processed corresponds to the time at which there is an onset of P-wave in ECG signal, for restricting the search space of guidewire detection, frames from several previous onset of P-wave is selected and their corresponding guidewire detection results are used. Frames corresponding to the same phase of the heart-beat need not always correspond to similar shapes of the guidewire. This is due to the fact that apart from motion due to heart-beat, there is also an effect of breathing of the subject that is seen in the video. The motion due to breathing is usually quite slow compared to that of motion due to heart beat. For this reason, an image processing based verification is done on the selected frames. All the frames in which the geographical location of the guidewire (after aligning the end points detected during initialization) correspond to significantly high tube-likeliness metric in the current frame is selected as a valid frame for search space reduction and other frames (which belong to the same phase of the heart beat but does not pass the tube-likeliness criterion—referred to as ‘invalid’ frames in the remainder of the paragraph) are discarded. In another embodiment, compensation for breathing is done for all ‘invalid’ frames as defined above. Mean of detected guidewires in the ‘valid’ frames is computed and is marked as reference guidewire. Point correspondence between the detected guidewires in the ‘invalid’ frames and the reference guidewire is computed as explained further herein. This point correspondence in effect nullifies the motion due to breathing in several phases of the heartbeat. Since this process separates the motion due to heart beat from motion due to breathing, it can be used further to study the breathing pattern of the subject:
The information of guidewire's location and shape in the previous frame allows us to narrow down the search range of the guidewire in the current frame.
The tube-likeliness metrics computed in the current frame for pixels in the region of interest
The guidewire end points in the current frame that is detected based on the guide catheter tip and the radio opaque distal part of the guidewire

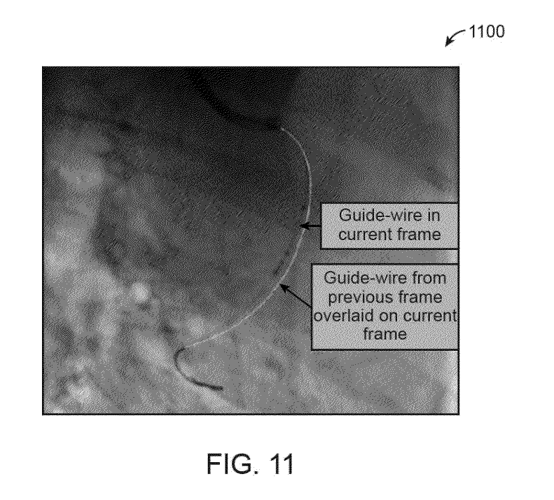

To narrow the search range for detecting the guidewire, the detected guidewire in the previous frame is mapped on to the current frame. Since the end points of the guidewire are known for the present frame, the previous frames guidewire is rotated scaled and translated (RST) so that the end-points coincide. Thus aligned image 1100 of the guidewire from the previous frame is mapped on the current frame as shown in FIG. 11.

It can be noted that the search space for the finding the present guidewire reduces tremendously once an initialization from the previous frame is taken into consideration. The prediction of the position of the guidewire can be made even better if the periodic nature of the change in trajectory due to heartbeat is taken into account. This however is not an essential step and each frame can individually be detected without using any knowledge of the previous frame's guidewire. The detection of guidewire after one complete phase of the heart-beat can consider the guidewire detected in the corresponding phase of the previous cycle of heart beat. Since the heart-beat is periodic and breathing cycles are usually observed at a much lesser frequency, the search-space can be reduced even further. The same phase of the heart-beat can be detected by using an ECG or other vital signals obtained from the patient during the intervention. In cases where vital signs are not available for analysis, image processing techniques can be used for decreasing the search space considerably. Analysis of the path of the endo-lumen device for significant amounts of time shows that the movement is fairly periodic. By selecting frames which have guidewires close to the regions of high tube-likeliness metric in the current frame, there is a high probability of selecting the correct frames for choosing the search space. To a fair extent, the correct frame can also be chosen by prediction filters such as Kalman filtering. This is done by observing the 2-D shape of the guidewire and monitoring the repetition of similar shape of guidewire over time. A combination of these two approaches can be used for more accurate results.

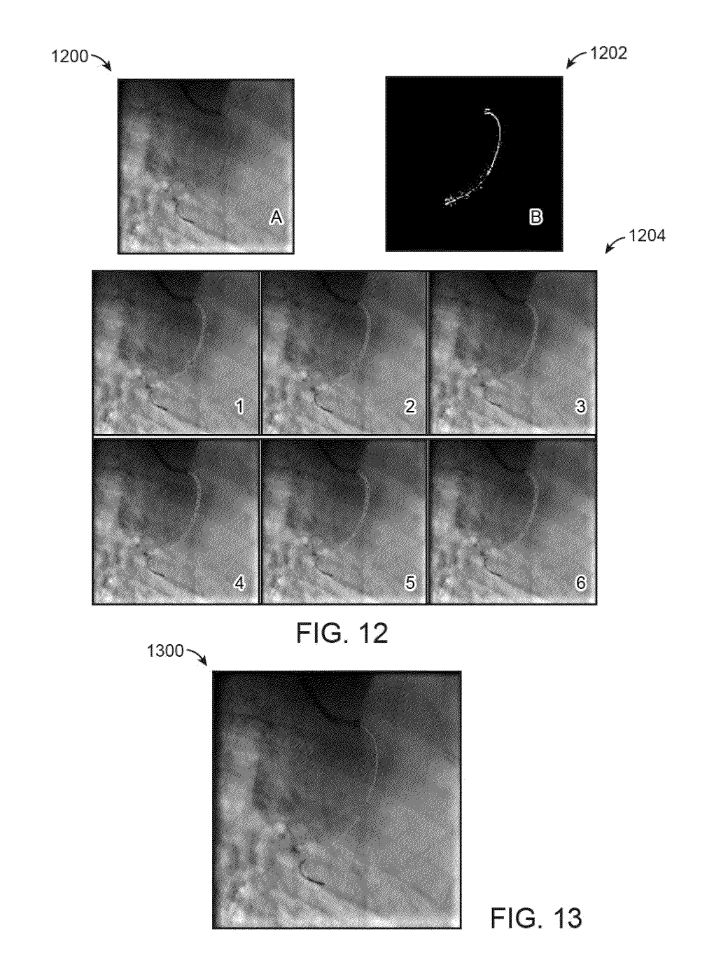

As evident in the FIG. 10, a number of discontinuous edges exist along the actual guidewire. The results of successive refinement of the detected guidewire are shown in the sequence of images shown in FIG. 12. The refinement shown is based on maintaining the continuity of the curve. In this Figure, the image 1200 of FIG. 12(A) is the raw image to be processed. Image 1202 of FIG. 12(B) is the tube Likeliness metric calculated for the image. Images 1204 of FIG. 12 numbered 1 through 6 represent identification of points on the guidewire with successive refinement. The final image (image 6) represents the final identification of points on the guidewire. Cubic spline fitting is then used to delete outliers and fit a smooth curve 1300, as shown in FIG. 13. Direct spline fitting in a noisy data would result in unwanted oscillations. Hence a spline fit with reduced degrees of freedom was used in our implementation.

Radiopaque Marker Detection and Guidewire Re-Estimation

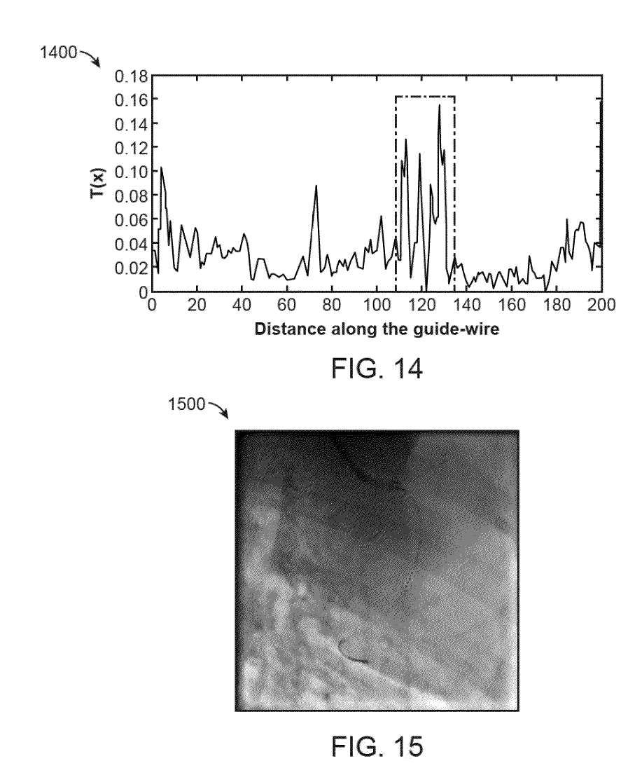

Markers being tubular in nature are often associated with high tube-likeliness metric. Hence, for localizing electrodes, we consider the T(x) values along the guidewire and detect numerous maxima in it. Contextual information can also be used to detect markers. If our aim is to detect balloon markers of known balloon dimensions, say 16 mm long balloon, the search for markers on the detected guidewire can incorporate an approximate distance (in pixel). Thus the detection of markers no longer remains an independent detection of individual markers. Detection of closely placed markers, such as the radiopaque electrodes used for lumen frequency response, can also be done jointly based on the inherent structure of electrodes. FIG. 14 shows a plot 1400 of tube-likeness values of the points on the guidewire. Significant maxima in such a plot usually are potential radiopaque marker locations. This plot 1400 also illustrates the procedure of detecting the inherent structure of the markers under consideration.

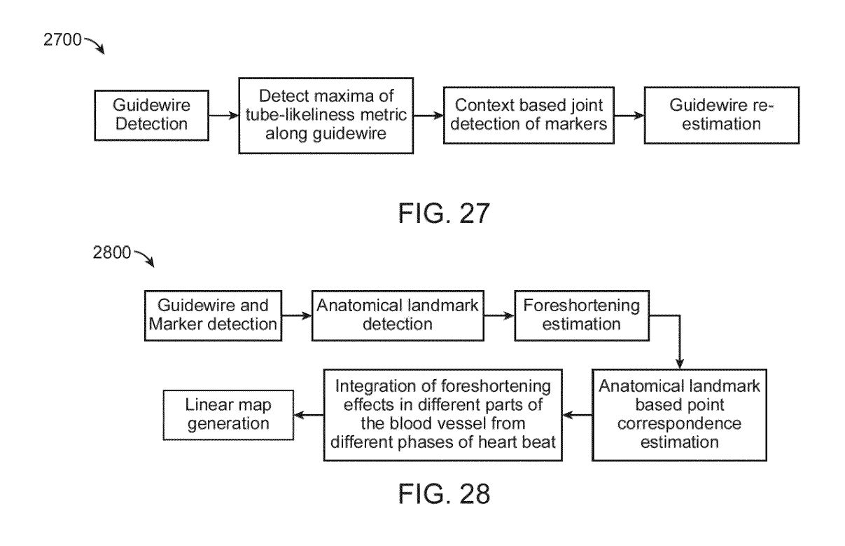

Since the markers are quite prominent structures in an endo-lumen device, the estimated marker locations are considered more reliable, if the detected path of the guidewire does not coincide with the center of the detected markers. In such cases a weighted spline fit algorithm is used to arrive at a better estimate of the guidewire, where markers are given a significantly higher weighting compared to the other points in the guidewire. This is because the markers, having strong features, are more reliably detected than the core of the guidewire. FIG. 15 depicts markers 1500 detected in the image. FIG. 27 shows a block diagram 2700 illustrating the different blocks of the marker detection algorithm. The location of markers is output number 5 as seen in FIG. 18 which illustrates an example of a block diagram enlisting various modules along with the output it provides to the end user.

In the discussion so far, we have assumed that the entire guidewire is a visible in the X-ray image. However, in some situations, the guidewire is not clearly visible in the X-ray image. This could be because of the poor quality of the X-ray image, low intensity radiation levels being used, or because of the material of the guidewire itself. In these cases, very few points (corresponding to the location of the markers in the endo-lumen device) on the guidewire would show up in the tube likeliness metric map. Guide catheter tip could be used as an additional point of reference. In such situations, only the path between the reliably detected points (markers and guide catheter tip) is estimated using the current frame. Motion compensation algorithm as discussed in the previous section is then applied to the partial guidewire section. As the markers are moved longitudinally along the artery, more segments of the guidewire are estimated. The information of the estimated guidewire segment is propagated both to the subsequent as well as previous frames. This helps in progressively detecting larger segments of the guidewire as the markers are moved and use the information of the trajectory of the markers to build the path of the guidewire. This process would help in building the guidewire path (and thus later create linear map) only till the point the markers progress. But as the markers (active electrodes/balloon markers in catheters) are usually taken at least up to the point where the stenosis occurs, partial generation of linear path would be sufficient for treatment planning and other interventional assistance.

Reduction of Search-Space for Automatic Segmentation/Localization

Reduction of search-space for automatic segmentation/localization of interventional tools (e.g., guidecatheter tip, guidewire tip, guidewire) may be based on future or past angiograms. Segmentation and detection of guidewire or interventional instruments such as stent catheters, IVUS catheters are needed on a continuous basis. This has many challenges including lower quality of X-ray, presence of other similar features in the image, fading of certain sections of the instrument, and computational complexity of searching large areas of the image. In order to mitigate some or all of these challenges, the angiogram can be used.

Image processing analysis of the angiogram yields the various arterial paths, from which the arterial path of interest is determined. A small localized region surrounding the arterial path of interest is selected as the search region for the instrument of interest. This reduced search region improves accuracy because it eliminates other prominent features that may lead to false detection. It also improves efficiency because of a smaller search space.

The shape of the interventional tools such as a guidewire changes with heartbeat and breathing of the subject. The reduction in search space is the best when the chosen angiogram belongs to the same heartbeat as well as breath phase. But, angiograms from other breath and heartbeat phases can also be used for reducing the search space for detection purposes. Further, there is a need for compensation for movement due to heart cycle. For this, the angiographic image corresponding to the same phase of the heart cycle is considered. There is also a need for compensation for breathing. This is achieved by matching the two end points that are usually reliably detected without the help of an angiogram—the tip of the guide catheter and the distal radio-opaque section of the guidewire—with the angiogram by a translational and rotational transformation such that the end points are made to lie on the identified arterial path of the angiogram.

Note that in some cases, the angiogram is recorded after the instrument is inserted. In such cases, the detection of the instrument can be done after the angiogram is recorded. There is no restriction on the sequence of events.

Guidewire Segmentation Using Joint Optimization Across Time

Segmentation of the guidewire or a similar endo lumen instrument is challenging in situations where the contrast in the X-ray is not high enough. In some cases, the instrument is barely visible and some sections of it may be completely invisible. During the heart cycle, the instruments undergo movement and change in shape, which often leads to different sections of the guidewire being visible in different image frames. Thus, even though there may not be enough detectable information about the guidewire in a single X-ray image, there may be enough information available over a series of images to do a robust detection. This can be done for example using a model for the guidewire with restrictions on its shape smoothness and deviations from the positions indicated by the angiogram and/or changes between successive image frames. An optimization program that jointly fits a guidewire model across time uses the following criteria and weighs them appropriately before selecting the optimal detected guidewire across time:
Match with segments that have a high likelihood of being part of the guidewire in each frame.
Smoothness of the model within a frame. This can be parametric (e.g. spline fit), or non-parametric, where constraints on local smoothness of the model in terms of its first, second and higher order derivatives
Constraints on deviation of the model guidewire across frames
Constraints on the deviation of the model wire in each frame from the corresponding angiogram at the same phase of the heart cycle

The constraints can be imposed as weighted penalties and an overall optimal model for each image frame is selected. This results in a more accurate detection of the guidewire in all frames compared to detecting it independently in each image frame.

Detection of Injection of Dye

The injected dye typically comes to the blood vessel of interest through the guide catheter tip. When the tip is being tracked automatically by an algorithm, presence of dye if gone undetected might result in completely bizarre results for guide catheter detection. FIG. 44 shows in image 4400 a dye being injected into an artery during a cardiac intervention. It can be seen that the characteristic pattern in a guide catheter tip goes completely missing when dye gets injected as shown in image 4402.

For detecting whether a dye is injected or not through image analysis, a region around the guide catheter tip is selected and continuously monitored for sudden drop in the mean gray level intensities. Once the drop is detected, it is confirmed by computing tube likeliness metric around the same region for highlighting large tube like structures. Presence of high values of tube likeliness metric around the region is taken as a confirmation for detecting a dye.

Guide catheter tip provides a good starting point for segmentation of the lighted up vessel as well. In literature, various complex seed-point selection algorithms exist. By tracking guide catheter tip, automatic detection of injection of dye and segmentation of lighted up vessel becomes possible. In theory, a detected guidewire, radiopaque markers, detected lesion, or any significant structure detected in the vessel of interest can be used as seed point for automatic segmentation of the vessel or for automatic injection of dye detection. It can also be detected automatically by connecting a sensor to the instrument used for pumping the fluid in. Such a sensor could transmit signals to indicate that a dye has been injected. Based on the time of transmission of such a signal and by comparing it with time stamp of the received video frames, detection of dye can be done.

Skeleton of Blood Vessel Path

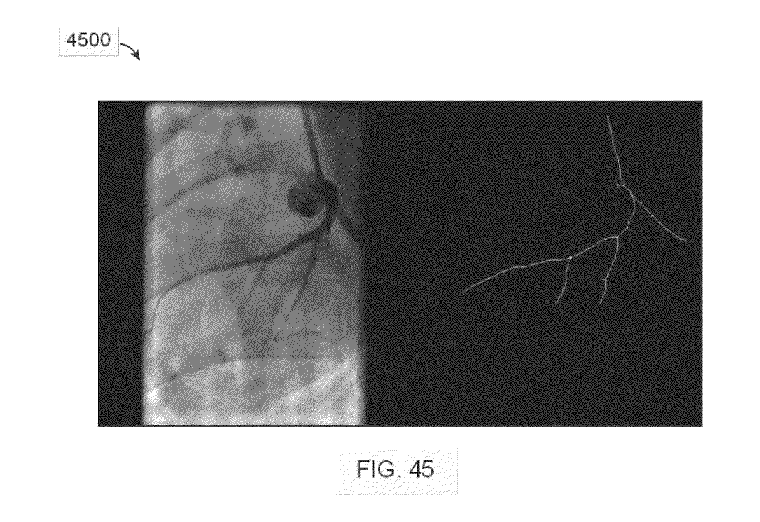

Skeletonization of the artery path, once a dye is injected can be done in multiple ways. Region growing, watershed segmentation followed by morphological operations, vesselness metric based segmentation followed by medial axis transform are some of the algorithms which could be applied. In our implementation, we use vesselness metric to further enhance the regions highlighted by the dye. A simple thresholding based operation is used to convert high tubular valued pixels to whites and the rest to black as seen in the adjacent images 4500 of FIG. 45 which illustrates the skeletonization of the blood vessel path. Selection of the threshold is an important step which enables us to select the regions of interest for further processing. We use an adaptive threshold selection strategy. This is followed by connected component labeling enables the selection of largest island of white pixels, connecting the region near a guide catheter tip. Medial axis transform gives a single pixel wide blood vessel path output. Branches, if any, also get highlighted using this operation. Any point from where a significantly large branch gets separated is detected by analyzing the neighborhood of each point in the detected skeleton. The location of branches is output number 4 as seen in FIG. 18 and it is used as anatomical landmarks to compensate for significant guide catheter movement.

Selection of Artery of Interest Based on the Guidewire/Guidewire Tip Detection

In a fluoroscopic image of the heart region taken during intervention, the tip of the guide catheter and distal radio-opaque section of the guidewire are clearly visible. These can be detected automatically or by some degree of user assistance. In either case, the detected pair of locations delineates the end points of the coronary arterial path that is of interest for the medical practitioner. Using these end points in conjunction with a static image of the angiogram, the full extent of the arterial path of interest can be identified automatically without any assistance from the user. Alternatively, if additional sections of the guidewire are identified, there would be enough information available to automatically detect the arterial path even without detecting the tip of the guide catheter. The steps can be summarized as follows:
1. In a fluoroscopic image in which a guidewire has been inserted, the guide catheter tip, the distal radio-opaque section of the guidewire are identified using methods already disclosed. Alternatively, at least a subset of the guidewire is detected.
2. A reference angiogram is selected for use by the medical practitioner. This could be selected automatically using methods disclosed later in this disclosure, manually, or by other means known in the art.
3. Using image processing algorithms already disclosed, the angiographic image is processed to segment the various network of branches that are lit up by the injected dye. This also yields all the possible arterial paths that could be of interest (candidate paths).
4. A subset of locations identified in step 1 are matched with the candidate paths identified in the previous step, and the best matching path is selected as the arterial path of interest.
5. Optionally, a compensation for motion due to heart beat and breathing is performed for the detected angiogram or the guidewire/guide catheter sections in order to improve the accuracy and robustness of the algorithm

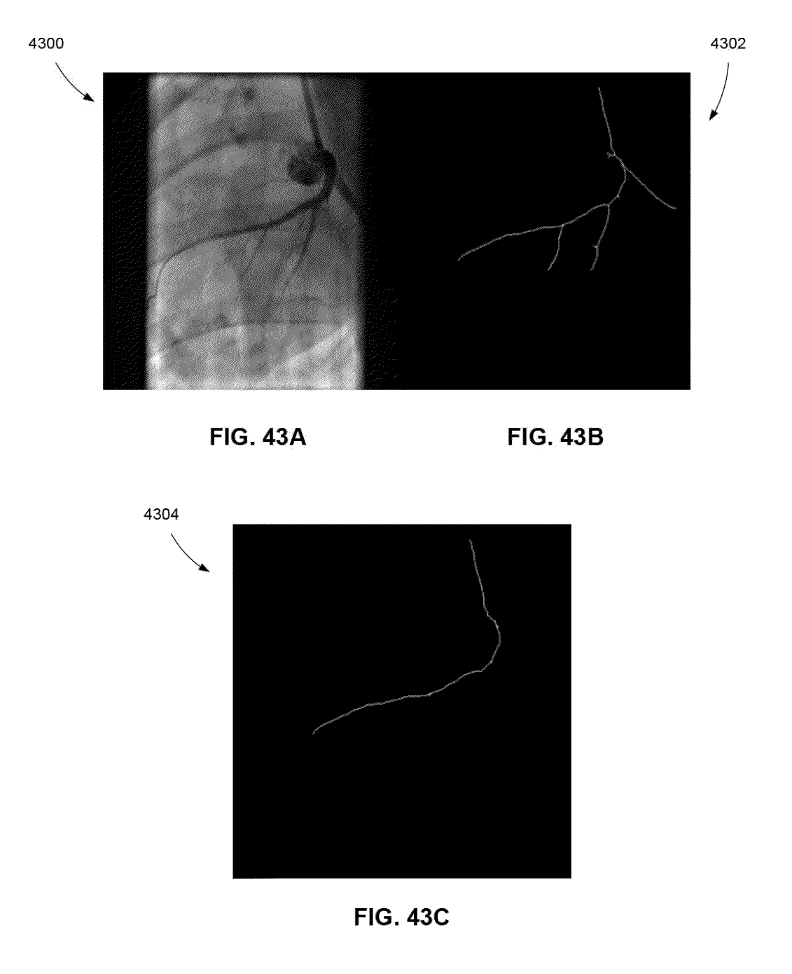

FIG. 43A shows a raw frame 4300 during an angiogram while FIG. 43B shows the identified highlighted regions/artery skeleton 4302. FIG. 43C shows the selected skeleton of artery of interest 4304 based on the guidewire tip detection in the artery. This can be used to trigger any image based lumen estimation algorithms known as QCA algorithms.

Selection of Static Reference Angiogram

Selection of static angiograms may be based on i) X-ray quality ii) percentage of artery of interest highlighted iii) length of branches. When an X-ray is recorded after injecting a dye, the resultant angiogram lasts for several frames of images before the dye fades out. Typically, the medical practitioner reviews all the candidate image frames before deciding on one specific image to be used as the reference angiogram. This method can be automated using an algorithm. The factors to be considered when deciding the optimal image are:
1. Quality of the X-ray: This is determined by analyzing the contrast present in the image. Noise analysis in flat sections of the image is also performed. Images with higher contrast and lower noise are preferred. Alternately, it can also be measured based on radiation intensity as denoted in the live video or in a DICOM tag.
2. Extent of arterial path of interest highlighted in the X-ray image: The arterial path of interest is identified manually or automatically on each candidate image frames. Each candidate image could have the radio opaque dye highlighting different extents of the arterial path and by different degrees of contrast. An image frame with stronger and fuller highlighting of the arterial path of interest is preferred
3. Length of branches: in case, there are multiple frames that highlight the artery of interest sufficiently, we choose the one which highlights the entire arterial tree structure including the branches sufficiently

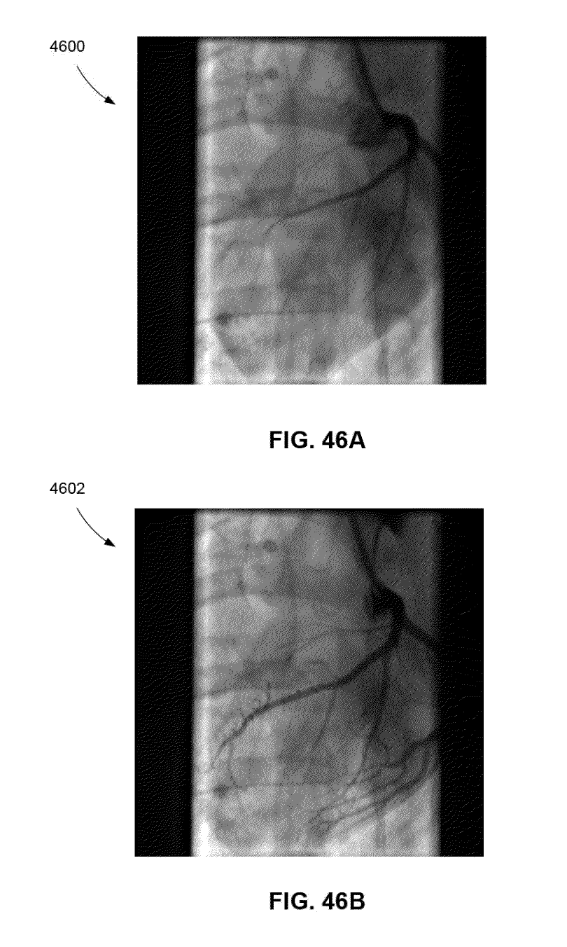

Both FIGS. 46A and 46B show angiograms which highlight the artery of interest. FIG. 46A highlights the artery of interest 4600 partially whereas FIG. 46B highlights 4602 it completely. By analyzing the sequence of images for the aforementioned parameters, the image on 4602 is chosen as the static angiogram. Apart from this, if the static angiogram is to be chosen for a specific purpose, such as for co-registering interventional tools during a motorized pullback or stent deployment, other factors may influence the selection of static angiogram selection as well. For example, in an ECG gated X-ray mode during motorized pullback of IVUS catheter, the phase in which X-ray is turned on can influence static angiogram selection.

Minimizing the amount of dye injected during a procedure is desirable as the radiopaque dye is known to be harmful for the kidney. Based on the selected candidate frames where the entire artery is highlighted, the least amount of dye that is required to highlight the artery of interest, in terms of fraction of the presently injected amount, can be evaluated. This can be communicated back to the interventionalist so that amount of dye injected can be minimized for all further injections including the present and future interventions performed in the artery of the patient. Moreover, if a lesion is marked manually in an angiogram, by analyzing the past angiograms, a decision regarding minimum amount of dye needed to highlight the lesion in the artery of interest can be taken, instead of the entire artery. This can further decrease the amount dye injected.

Blood Vessel Diameter Measurement

On either side of the detected skeleton, a normal is drawn (perpendicular to the direction of the tangent at that location). Along the direction of normal, derivatives of gray level intensities are computed. Points with high values of derivatives on either side of the skeleton are chosen as ‘probable’ candidate points for blood vessel boundaries. For a single point in the skeleton, multiple ‘probable’ points are selected on either side of the contour. A joint optimization algorithm can then be used to make the contour of the detected boundaries pass through maximum possible high probable points without breaking the continuity of the contour. Alternately, only the maximum probability point can be chosen as boundary points and a 2-D smoothing curve-fitting algorithm can also be applied on the detected boundaries so that there are no ‘sudden’ unwanted changes in the detected contours. This is done to get rid of the outliers in the segmentation procedure.

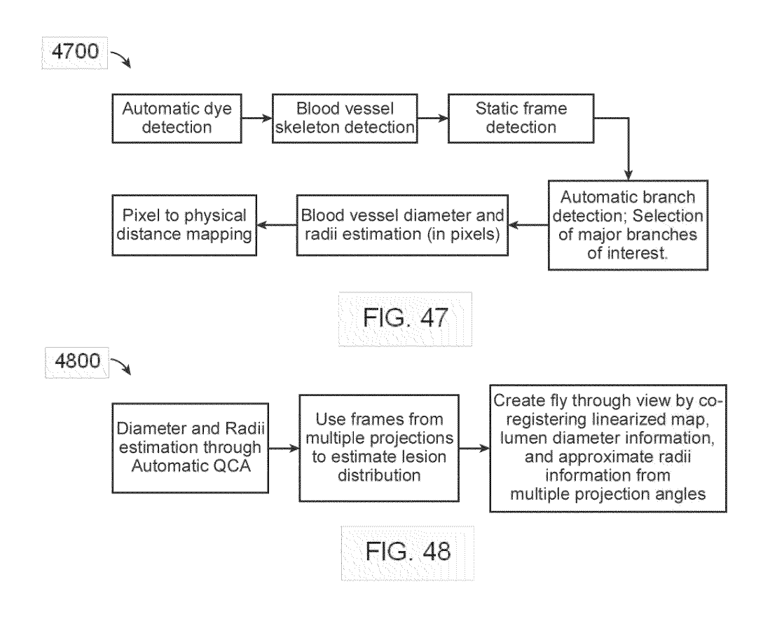

In a normal use case scenario, injected dye progresses gradually within the vessel. Progressively more and more of the vessel gets lighted up in the X-ray. In such a case, a several parts of the vessel may get lighted up in different frames of the video. It is not mandatory for the entire vessel to get lighted up in the same frame. In such a case, the above described joint-optimization algorithm can easily be extended to multiple frames. In cases where similar parts of the artery gets lighted up in multiple frames, joint optimization and estimation will result in more robust estimation of diameter. Similar parts of the artery can be detected using the anatomical landmarks based point-based correspondence algorithm discussed previously herein. Also shown in the block diagram 4700 illustrating an automatic QCA algorithm in FIG. 47.

The distance between 2 corresponding points along the normal of a particular point in the skeleton would give us the diameter of the blood vessel. The difference in the radius along either side of the normal would give us an idea on any abnormally small radius on either side of skeleton. This might in turn aid in detecting which side the lesion is present. Marker positions in different locations along the blood vessel, if present, can be used in aiding the conversion of Automatic QCA results from pixels to millimeter. If these are not present, diameter of the guide catheter tip can be used as a reference for the conversion. The QCA results for blood vessel only serves as an approximate estimate of the diameter since it works on a single 2-D projection. It can act as a good starting point for any lumen diameter estimation algorithms such as OCT, IVUS or the one explained herein. This is output number 1 as seen in FIG. 18. QCA estimate can also be used as a feature for obtaining a good point correspondence as described later.

Lumen diameter estimation when co-registered with a linearized view of the blood vessel would give us an idea regarding the position of a lesion along the longitudinal direction of the blood vessel. However representation of a skewed lesion with the diameter alone can sometimes be misleading. Estimation of left and right radii along the lumen helps in visually representing the co-registered lumen cross-sectional area/diameter data accurately. Alternately, linear scale as generated with the linearization technique can be co-registered on the image with accurately delineated blood vessel to represent QCA and linearized view together.

If Automatic QCA is computed in multiple 2-D projections, it can be combined with the 3-D reconstruction of the blood lumen trajectory (as explained herein). Combination of the two also helps in creating a fly-through view of the blood vessel. Fly through data can also be computed without resolving the ambiguity of 3-D reconstruction (as explained herein). This is output number 3 as seen in FIG. 18 and as also shown in the block diagram 4800 of FIG. 48 which illustrates a fly-through view generation algorithm. The 3-D reconstruction along with lumen diameter information can be used for better visual representation of the vessel and can be used as a diagnostic tool during intervention as well.

Apart from automatic QCA, injection of dye is also quite useful in detecting guide catheter tip automatically as mentioned herein. Since detection of guide-catheter tip is almost a necessity for all further steps in linearization, injection of radiographic fluid whenever angle of the C-arm machine is changed becomes quite useful. If this becomes too much of an overhead for the interventionalist, dye can be injected only in the final view (after the placement of endo-lumen device such as the guidewire), before the placement of stent. This would enable the algorithm to go seamlessly to the ‘guidance’ mode as described herein.

Point Correspondences (Motion Compensation), Co-Registration and Linear Map Generation

When co-registering an object that is detected in a fluoroscopic on to a reference angiogram, there are several motion artifacts that need to be compensated for such as motion due to heart beat, motion due to breathing, translationary movement of the patient, zoom in camera. The compensation for all of these are done is a two-step process.
1. The angiographic image corresponding to the same phase of the heart cycle as the current fluoroscopic image to be co-registered is selected. These two images would differ in viewed content due to movements other than heart cycle (breathing, translation, zoom). This movement is compensated by estimating the amount of translation, zoom and rotation around an axis that is perpendicular to the image plane (methods described in previous disclosures). After compensation, the objects in the current fluoroscopic image are co-registered on to the selected angiographic image
2. The selected angiographic image need not be the same as the single reference angiographic image selected for co-registering for all phases of the heart cycle. In order to co-register on to this reference angiographic image, motion that is purely due to heart cycle is compensated for. This compensation method is already described using anatomical landmarks and geometrical landmarks.

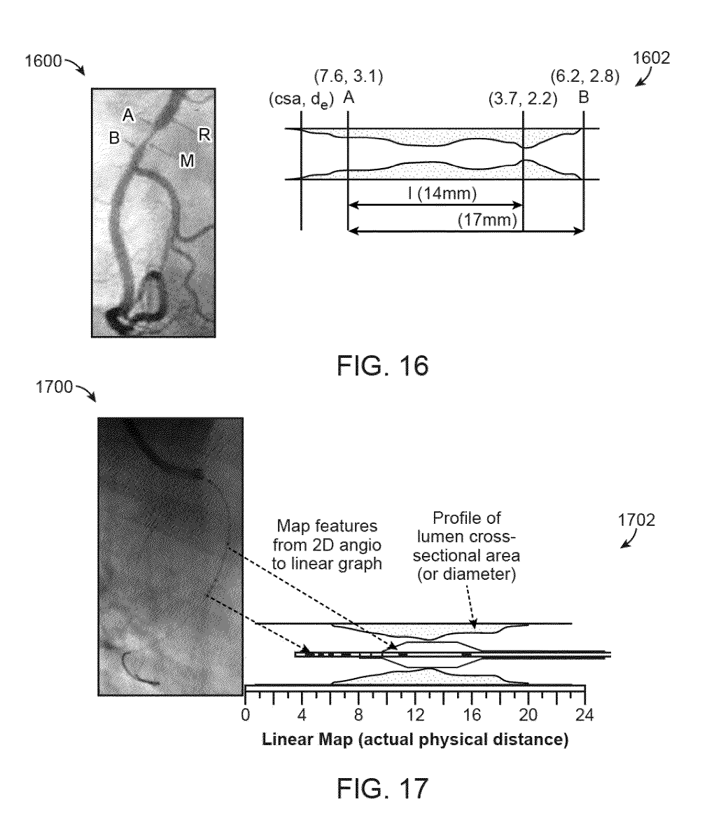

An example of the linear map generation is depicted in FIG. 16 which illustrates the linearized path 1602 co-registered with the lumen diameter and cross sectional area information 1600 measured near a stenosis. After detecting the radiopaque markers on an endo-lumen device, distance between them can be measured along the endo-lumen device (in pixels). Knowing the physical distance between these markers helps in mapping that part of the endo-lumen device into a linear path. If there were closely placed radiopaque markers present throughout the endo-lumen device, a single frame is enough to linearize the entire blood vessel path (covered by the endo-lumen device). The radiopaque markers needs to be placed close enough to assume that path between any two consecutive markers are linear and that the entire endo-lumen device can be approximated as a piecewise linear device.

Note that the mapping between pixels and actual physical distance is not unique. This is because the endo lumen device is not necessarily in the same plane. In different locations, it makes a different angle with the image plane. In some locations it may lie in the image plane. In other locations it may be going into (or coming out of) the image plane. In each case, the mapping from pixels to actual physical distance would be different. For example, if in the former case, the mapping is 3 pixels per millimeter of physical distance, for the latter it could be 2 pixels per millimeter. This physical distance obtained gives an idea of the length of the blood vessel path in that local region.

In an actual use case scenario, placing many radiopaque markers in an endo-lumen device may not be useful for the interventionalist as it might obstruct the view of the path and possible lesions present in them. Thus there is a need to minimize the number of markers placed on the endo-lumen device. The other extreme case is to place a single marker on the endo-lumen device. This would allow us to track the marker in all the frames. If the marker is of known length, the variation of the length of the marker in different locations along the lumen can be used for creating a linearized map. In case a single marker of a significantly small length (where it can no longer be approximated as a line but as a single point on the image) is used, a calibration step of having a motorized pullback is required. This would allow us to map different points in the blood vessel to different points in the linearized map. This will minimize the view obstruction constraint of an interventionalist but at the same time, it adds an additional step (of motorized pullback) for getting the same result. Hence as per our analysis, 2 to 5 closely placed markers near the distal end of the endo-lumen device is an optimum design for aiding an intervention by creating a linearized path. When such an endo-lumen device is inserted, by analyzing multiple frames, as and when the endo-lumen device is pushed in, a linearized view of the blood vessel can be created. It should be noted that for the invention described here, the distance between adjacent markers need not be small enough for the assumption—that they are in the same plane—to hold true. In cases where the distance is large, such as that in balloon markers, distance between corresponding markers in consecutive frames is measured after motion compensation and this distance is further used for linearization.

The observed motion in an imaged frame could be a result of one or more of the following occurring simultaneously: translation, zoom or rotational changes in the imaging device; motion due to heart-beat and breathing; physical motion of the subject or the table on which the subject is positioned. The shape or position of the blood vessel is going to be different in each phase of the aforementioned motion. Thus linearization of the blood vessel is no longer a single solution but a set of solutions which linearizes the blood vessel in all possible configurations of the motion. But such an elaborate solution is not required if the different configurations of the blood vessel is mapped to one another through point-correspondence.

Finding correspondences between corresponding structures has been an extensively researched topic. Image-based point correspondences may be found out based on finding correspondences between salient points or by finding intensity based warping function. Shape based correspondences are often found based on finding a warping function which warps one shape under consideration to another and thus inherently finding a mapping function between each of its points (intrinsic point-correspondence algorithms). Point correspondences in a shape can also be found out extrinsically mapping each point in a shape to a corresponding point in the other shape. This can be either be based on geometrical or anatomical landmarks or based on proximity of a point in a shape to the other when the end points and anatomical landmarks are overlaid on each other. Anatomical landmarks used for this purpose are the branch locations in a blood vessel as described herein. Landmarks that are fixed point on the device or devices visible in the 2-D projection such as tip of the guide catheter, stationary markers, and fixed objects outside the body may also be used. Correlation between vessel diameters (as detected by QCA also described herein) in different phases of the heart beat can also be used as a parameter for obtaining point correspondence. In our implementation, we have used extrinsic point-correspondence algorithm to find corresponding locations of markers in each shape. By finding the point correspondence between different parts of the endo-lumen device in different phases of the heart-beat, foreshortening effect estimated in one phase can be translated to other phase and thus helps in integrating the foreshortening effects. This is used in creating a linearized map of the entire path traversed by the endo-lumen device. FIG. 28 shows a block diagram 2800 of different blocks involved in the linearization algorithm.

Motion compensation achieved through extrinsic point-correspondence can be used for compensating all of the aforementioned scenarios. It also reduces the amount of computation required for motion compensation as compared to image-based motion compensation techniques. However, as mentioned earlier in the section, image-based motion compensation or motion prediction strategy may be used to achieve the same goal by using a dedicated high-speed computation device. The resultant motion compensated data (locations of endo-lumen devices in case of guidewire based motion compensation; image(s) in case of image-based motion compensation) can be used to compute translation of endo-lumen devices/markers along the longitudinal axis of a lumen. This computed information can further be visually presented to the interventionalist as an animation or as series of motion compensated imaged frames with or without endo-lumen devices explicitly marked on it. The location information of the markers and other endo-lumen devices can also be superimposed on a stationary image.

Co-registration may require compensation for movement of markers due to breathing compensation between the guidewire and artery in an angiogram (in the same phase of heartbeat), e.g., by use of geometrical landmarks. Heartbeat compensation between highlighted arteries (during angiogram) in different phase of the heartbeat may also be accomplished, e.g., by use of anatomical landmarks such as vessel branches.

Features of this co-registration algorithm may be used with any of the devices and methods as disclosed in the following patents and patent applications (which are incorporated herein by reference in their entirety and for any purpose herein) and in any possible combination:
U.S. application Ser. No. 13/159,298 filed Jun. 13, 2011 (US Pub. 2011/0306867 A1)
U.S. application Ser. No. 13/709,311 filed Dec. 10, 2012 (US Pub. 2013/0123694 A1
U.S. application Ser. No. 13/305,610 filed Nov. 28, 2011 (US Pub. 2012/0101355 A1)
U.S. application Ser. No. 13/305,630 filed Nov. 28, 2011 (US Pub. 2012/0071782 A1)
U.S. application Ser. No. 13/305,674 filed Nov. 28, 2011 (US Pub. 2012/0101369 A1)
U.S. application Ser. No. 13/764,462 filed Feb. 11, 2013 (US Pub. 2013/0226024 A1)
U.S. application Ser. No. 13/946,855 filed Jul. 19, 2013 (US Pub. 2014/0032142 A1)
U.S. application Ser. No. 14/078,237 filed Nov. 12, 2013 (U.S. Pub. 2014/0142398 A1)
U.S. Prov. App. 61/763,275 filed Feb. 11, 2013
U.S. Prov. App. 61/872,741 filed Sep. 1, 2013

Note that some rotational movements are well approximated as translation. For example, if the patient turns by a small amount. In all such cases, the same methods can be employed.

In cases where the linearizing is not possible/performed based on the inserted device, the information regarding direction of motion of the device, speed of motion along the longitudinal direction of the lumen (if known) can further be used for refining the coregistration. For example, if the pullback is a known constant speed, this a priori information can be used to correct for small errors in co-registration by imposing appropriate constraints such as smoothness. Further, knowledge of foreshortening angle can be used for even tighter constraints.

Linearization and Based on Markers Located at a Distance from One Another

Linearization based on markers that are close to each other has been described in [Ref]. Here, it is assumed that the segment between the markers is well approximated by a straight line segment. This implies that the fore-shortening effect is the same for all points in the segment between the two markers. However, this assumption is incorrect if the markers are further apart. In such a case, the segment between the end points could be a curve in 3D with different foreshortening at different parts of the segment. The segment takes the shape of the lumen trajectory through which it traverses. This is solved by a different method as described fore with

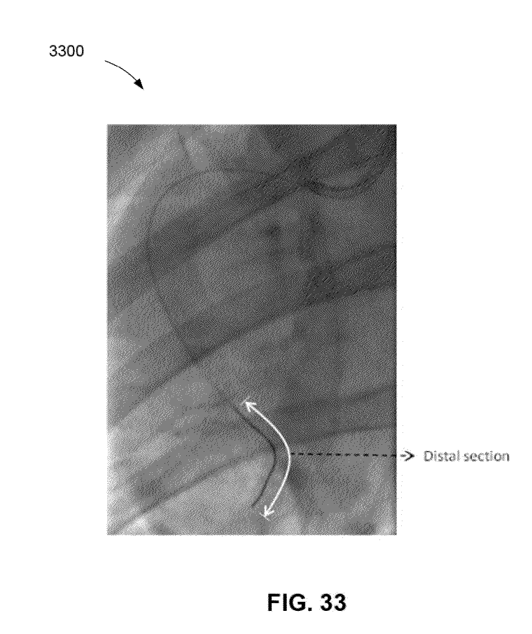

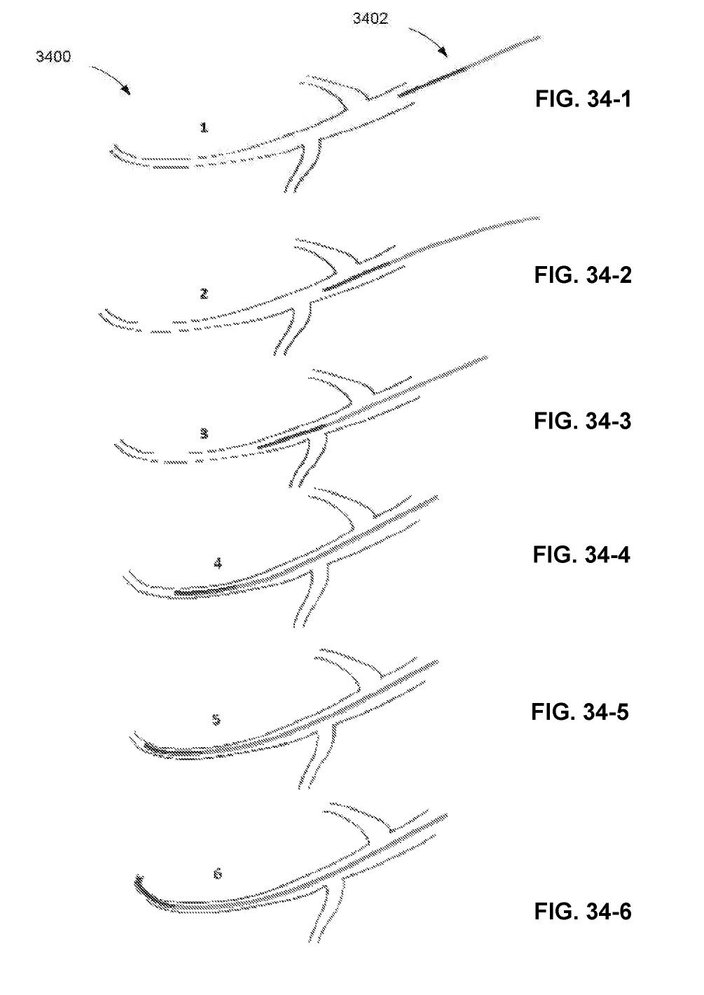

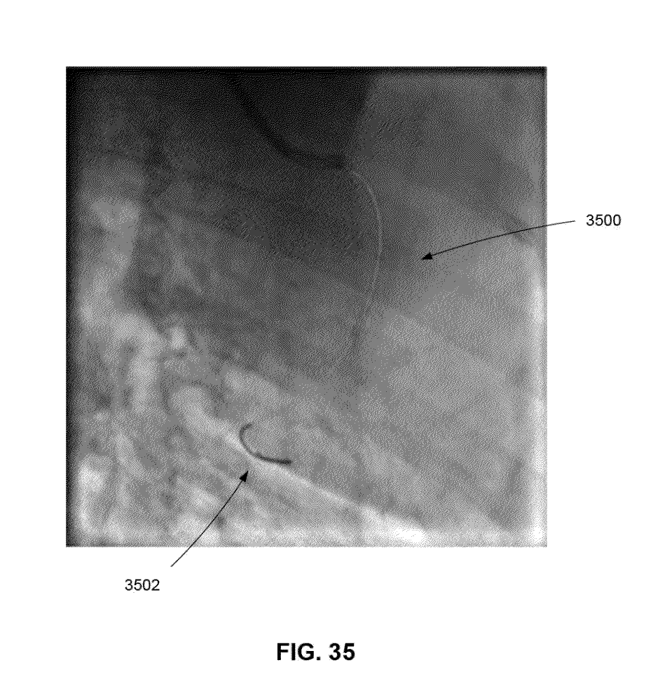

Linearization and 3D reconstruction may be based on at least two markers which are located at some distance from each other, e.g., balloon markers, IVUS markers based linearization, etc. In guidewires, the distal section of the guidewire, which is a few cm in length, is very clearly visible in an X-ray image 3300, as shown in FIG. 33. This section has a length that is known a priori. As shown in the example of FIGS. 34-1 to 34-6, a guidewire 3402 having a tip section may be inserted into an artery 3400. This distal section in its entirety traverses the region of interest in the artery before it reaches its final position. Using techniques described earlier, the tip section 3502 of the guidewire 3500 shown in FIG. 35 can be detected and the end points of the tip section 3502 identified clearly. These end points are equivalent to two markers that can be distinctly identified.

As this distal section traverses the trajectory of blood vessel, the apparent length of the section measured along the winding trajectory of the section changes as it moves along different locations of the blood vessel. Between successive image frames, the distal tip section would have moved by a small amount. The proximal end of this section and distal end of the section may move by different amounts in terms of pixels. This is because at different locations the trajectory would subtend different foreshortening angles with the image plane. These displacements of the two ends of the section are related to the foreshortening angles that the trajectory makes with the image plane at the respective ends. As the tip of the guidewire is continually tracked as it moves through the trajectory of interest, the relative foreshortening at each point in the trajectory Using the knowledge of the actual length of the tip section of the guidewire, and the relative foreshortening angles at each point in the trajectory of interest, the mapping of pixels to actual distance at each point in the trajectory is determined.

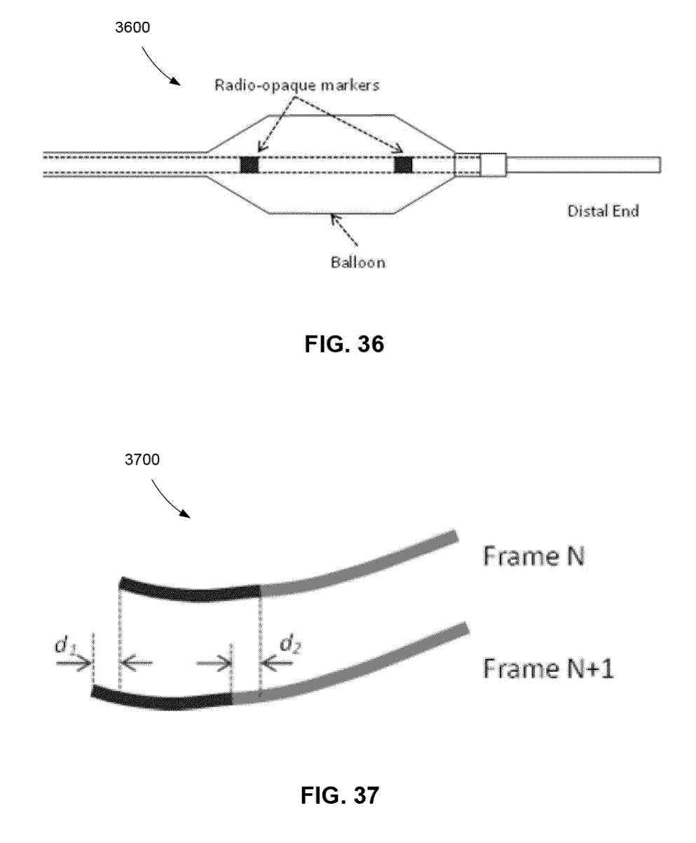

Similar to the tip section of the guidewire, there are other situations where there are two or more distinct points visible on a device that maintains a constant distance between them along the longitudinal axis of the device. Examples of these include balloon markers 3600 having two markers that are spaced apart by a known distance as shown in FIG. 36. The markers could also consist of distinctive features in a device such as IVUS. It could also be a shape that is clearly detectable at least in terms of its end points.

The description below is described for the tip of a guidewire. However, it is equally applicable for any device that has at least two points, such as the balloon catheter of FIG. 36, that are detectable and have a known distance between them along the axis of the device since only the positions of the two end points of the tip section are used for calculations.

In two successive frames ‘N’ and ‘N+1’ as shown in the diagram 3700 of FIG. 37, the guidewire translates along the trajectory of the blood vessel. The far end of the guidewire has translated by a small amount d₁, and the near end by an amount d₂, both measured in terms of pixels. These two measurements may not be equal and are related by the respective foreshortening angles, θ₁ and θ₂. The actual linear displacement of the two end points along the trajectory of the blood vessel, L, is the same at both ends since the actual length of the distal section does not change (it is rigid along its axis and cannot be stretched or compressed). The linear displacement D is related to the observed displacement and foreshortening angles by the following relation:

$$D = \frac{{Kd}_{1}}{cos ( {θ}_{1} )} = \frac{{Kd}_{2}}{cos ( {θ}_{2} )}$$

Where K is a constant that maps pixels to distance, and is determined by pixel density and zoom factor of the camera. Thus we have:

$$\frac{{d}_{1}}{cos ( {θ}_{1} )} = \frac{{d}_{2}}{cos ( {θ}_{2} )}$$

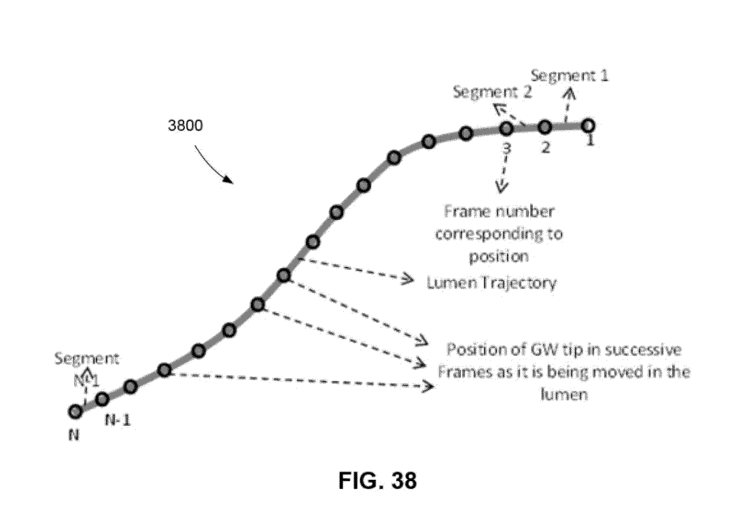

As the tip section of the GW moves through the trajectory of the lumen, the tip of the guidewire moves through different points on the trajectory over successive frames. The set of ‘N’ points it moves through is depicted in FIG. 38.

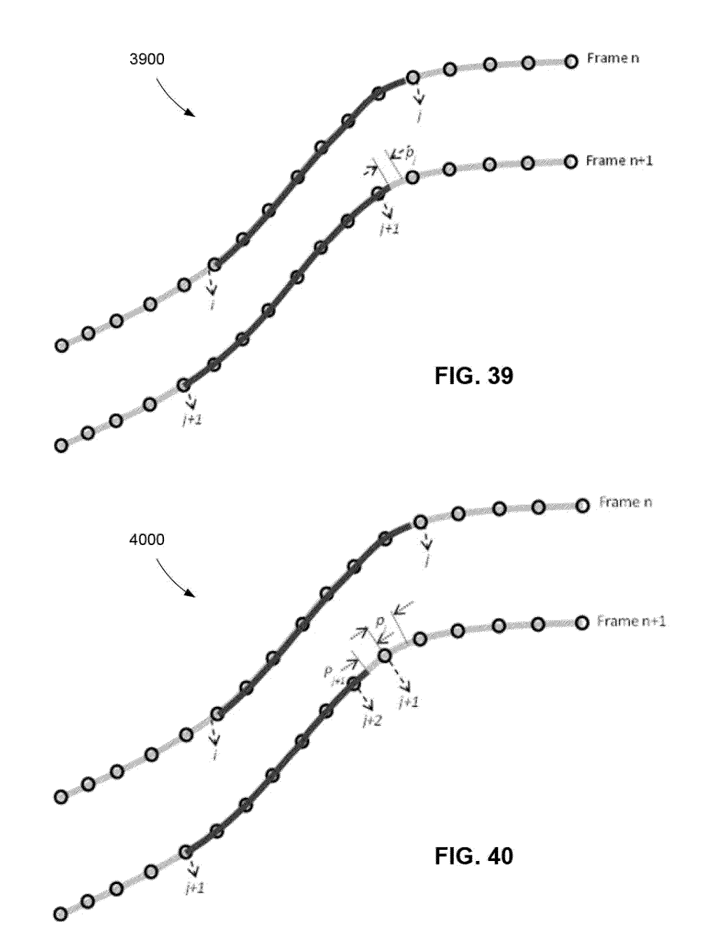

Similarly, the proximal end of the guidewire tip section would traverse through several points over successive frames. These points need not coincide with the points through which the distal end traverses. For simplicity, initially assume that the guidewire tip is moved slow enough that the points through which it traverses are a set of points that are close enough to define a piece-wise linear set of segments that represent the trajectory. There are N−1 segments, each with an observed length, d₁, and corresponding foreshortening angle, cos(θᵢ). When the distal end of the tip section moves from point i to i+1, the proximal end would also have moved by a small amount. In general, the distal end in each frame would lie between two points. If the movement of the proximal end point is wholly contained in the segment j, i.e., between points j & j+1, the foreshortening angles at i & j are related by

$$\frac{{d}_{i}}{cos ( {θ}_{i} )} = \frac{{p}_{j}}{cos ( {θ}_{j} )}$$

where pⱼ is the distance moved by the proximal end point in terms of pixels (this is not the same as dⱼ). This case is depicted in FIG. 39.

In the cases where the movement of the proximal end point starts in segment j₁ and ends in j₂, the physical movement of the proximal end point is the sum of physical movements in the individual segments through which it traverses. For example, if it starts in segment j and ends up in segment j+1, then we have:

$$\frac{{d}_{i}}{cos ( {θ}_{i} )} = \frac{{p}_{j}}{cos ( {θ}_{j} )} + \frac{{p}_{j + 1 }}{cos ( {θ}_{j + 1} )}$$

where pⱼ and pⱼ₊₁ are pixel distances by which the proximal end point has moved. This case is depicted in FIG. 40.

If the proximal end point traverses through more than two segments, the number of terms in the RHS of the equation would correspondingly increase.

For establishing the first relationship between the foreshortening angles, the entire tip section would need to be visible. In this situation, the distal end point would be at position M, and the proximal end point would be somewhere in the first segment (between locations 1 and 2). Relationships between foreshortening angles can commence from this point onwards. Thus, by tracking until the distal end point reached location N, (N−M−1) independent relationships between the foreshortening angles would be known. These include all the unknown foreshortening angles (N unknowns) in the region of interest (alternatively, the guidewire tip can be detected and tracked over a larger section of the trajectory to get N sets of relationships). Since there are more unknowns than the number of equations, it is still not possible to solve the unknowns.

Additional set of equations are obtained by exploiting the fact that the length of the tip section is constant, i.e., it neither gets physically stretched nor physically compressed. In any given frame, the total length of the guidewire tip section is given by the sum of the segmented parts after correcting for foreshortening:

$$L = {\sum }_{i = k ( n )}^{i = k ( n ) + M ( n ) - 1} \frac{Q \cdot {l}_{i}}{cos ( {θ}_{i} )}$$

where,
n is the frame number
lᵢ is the length per pixel of the part of the GW tip section occupying the i^{th} segment,
M(n) is the number of segments occupied (wholly or partially) by the guidewire tip
k(n) is starting segment number
θᵢ is the foreshortening angle
Q is the scale factor to convert pixels to physical measurement units (e.g. pixels to mm)

By definition, lᵢ=dᵢ for all segments except the first (i=k(n)). This is because the first segment contains the proximal end point, and can be located anywhere within the segment. The value of M(n) can vary from frame to frame because of difference in foreshortening effects in different parts of the lumen trajectory. By applying the above equation for each frame, we get a further N−M(1) equations, where M(1) is the number of segments occupied by the guidewire tip in the first frame where the tip is wholly visible. Also note that all these equations are independent.

Thus, in all we have (N−M−1)+(N−M)=2*N−2*M−1 equations, and N−1 unknowns. If N is sufficiently larger than M, we would have at least as many independent equations as unknowns. But this is not sufficient to solve all the unknowns. There is one ambiguity still left. The scale factor Q always appears as a ratio with cos(θᵢ), i.e., γ(i)=Q/cos(θᵢ). Hence, both can be scaled without affecting the result. However, this ambiguity does not matter if one is only interested in linearization. This is because the linearization expression, which is conversion of observed pixel length to actual physical distance that is compensated for foreshortening, always has the two ambiguous quantities as the ratio γ(i), and there is no ambiguity in the calculated linearized value.

Nevertheless, there is still a way to resolve this ambiguity by exploiting a condition that is likely to be satisfied in most cardiac intervention procedures. In most procedures, the angle of the C-arm that captures the X-ray image is adjusted for optimal viewing experience. In this optimal viewing angle, it is reasonable to assume that there would exist a point somewhere in the middle of the lumen trajectory that is closest to the X-ray, and on either side of this point, the points would be further away. This point also corresponds to zero foreshortening, i.e., θᵢ=0. Since cos(θᵢ) cannot take a value larger than 1, the value of θᵢ that corresponds to the largest value of Q/cos(θᵢ) can be assumed to be zero, thus resolving the ambiguity.

It is important to have a large number of frames to get a more robust estimate. When N is significantly larger than M, then we have a highly over determined set of equations. A least squares estimate can be used to calculate the unknown variables. This gives robustness to various sources of errors such as identifying of end points.

Though the depictions suggest that the guidewire is moved in one direction, this is not a requirement. There could be repeated back and forth movements. These in fact would be preferable to get a dense set of samples of end points in the region of interest leading to a more robust estimate.

Note that in this description, the guidewire tip section was chosen as the device. The same method can be used to linearize the segment between two balloon markers which are sufficiently far apart such that the section of guidewire or catheter between the markers is visible, and is no longer well approximated by a straight line segment. Further, if there are more than 2 detectable points on the device, a similar approach can be followed. For example different pairs of points can be selected at a time and the same analysis can be performed for each selected pair. These results can be combined to give a final estimate that is more accurate. It is also possible to consider all or a subset of points simultaneously.

It should be further noted that once the fore-shortening angles are known, 3-D reconstruction can be done using methods described later in the document for the case when the markers are close to each other.

Differentiating Between Interventional Instruments from Prominently Visible Extraneous Objects/Features in an X-Ray

Differentiating between interventional instruments such as a guidewire tip and other interventional tools from prominently visible extraneous objects, e.g., ribs, CABG wires, etc., may be accomplished by studying their movement across different phases of heartbeat. In a fluoroscopic image, there are several features and objects that are typically visible. Some of these are important from the point of view of the interventional procedure. Examples are guidewire, guide catheter, stent catheter, stent, IVUS catheter and associated markers. The objects/features that are not important are the ribs of the patient, pacemaker, wires inserted after a CABG (bypass) procedure, instruments or objects lying near the patient within the field of X-ray. It is important to be able to distinguish between these two classes of objects/features in order to achieve robustness for any automated image processing algorithm that need to selectively detect the relevant objects and features. This may be achieved by the following process:
Detect any object/feature that is visible in the X-ray in multiple frames across the different phases of the heart cycle.
Determine the correlation of the position of the detected object/feature with the heart cycle.
Objects that are present in the arteries of the heart such as the guidewire or its sections, the tip of the guide catheter, balloon markers, stents, and IVUS catheters all move with the heart and follows the heart cycle. Thus these objects show a higher degree of oscillatory motion whose periodicity is correlated with the heart cycle. On the other hand, the other objects that are further away from the heart such as ribs, CABG wires and external objects show much lower correlation with the heart cycle. This difference in correlation is used to distinguish between the two classes of objects/features.

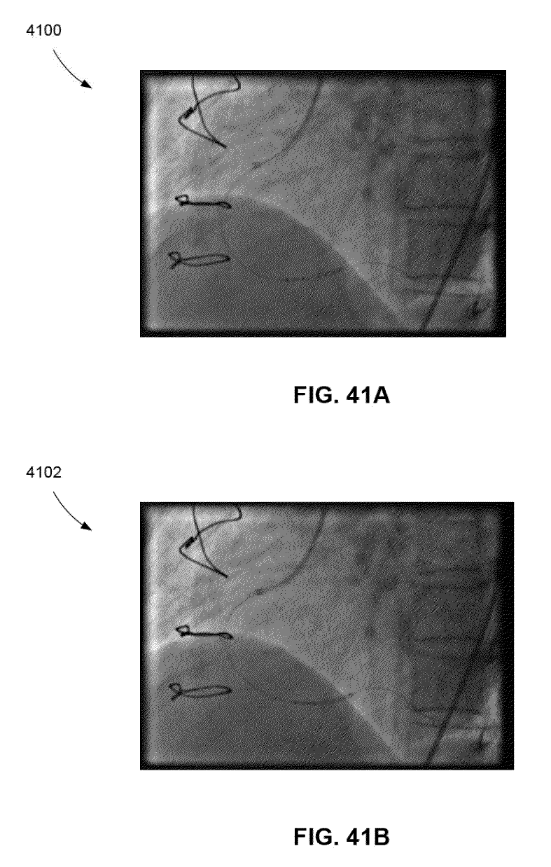

FIGS. 41A and 41B show images with CABG wires and interventional tools such as the guidewire in two different phases of the heartbeat. By analyzing the shape and position of these structures in multiple frames 4100, 4102 the interventional tools are differentiated from other prominent structures.

3-D Reconstruction

Each time a part of the endo-lumen device is linearized, angle it subtends with the 2-D projection plane can be measured based on the apparent foreshortening effect. But there is an ambiguity with respect to whether the part of the endo-lumen device comes out of the plane towards the x-ray receiver or goes away from it. This ambiguity cannot be resolved by this technique. Hence, when linearization of the entire endo-lumen device is done based on ‘n’ separate estimations of foreshortening effect in different parts of the blood lumen trajectory, each part gives a binary ambiguity with respect to 3-D reconstruction of the blood lumen trajectory. The ‘n’ separate estimations may be done based on multiple markers throughout the endo-lumen device or by any sub-sample of it or by any technique mentioned in the above section or by methods mentioned herein. Hence ‘n’ step linearization procedure will have 2ⁿ consistent solutions of 3-D reconstructions. However, not all solutions can be physically possible considering the natural smoothness present in the trajectory of the blood lumen. Several of the 2ⁿ solutions can be discarded based on the smoothness criteria. Further, using other information, such as the convex nature of the heart's wall, a unique solution to this ambiguity is possible.

It is a common practice during intervention to view a blood vessel from multiple angles before arriving at a decision. Linearization in multiple angles (at least 2 angles) helps in narrowing down the possibilities of 3-D reconstructed path down to one. This includes, detecting and tracking endo-lumen device and radiopaque markers, motion compensation followed by linearization in at least 2 angles.

In another embodiment, when the projection angle of the C-arm is changed, all possible 3-D reconstructed paths are projected to the new projection angle. Each reconstructed path will have a separate projected path in the new projection angle. Endo-lumen device is detected in the new angle too and all the predicted projections which do not match the detected endo-lumen device's path are rejected. By using projections in multiple angles, verification and narrowing down of 3-D reconstructed path can be done. This procedure helps in finding a 3-D reconstructed path of the blood lumen trajectory.

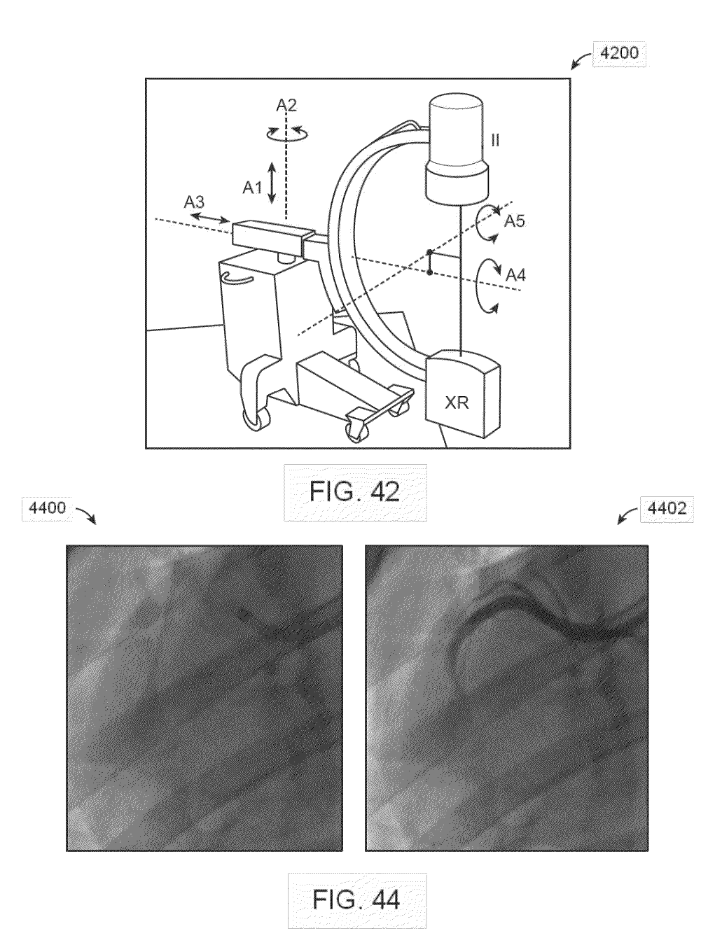

For obtaining a 3-D reconstructed view of the trajectory, the projection angle of the C-arm must be uniquely determined. The C-arm has 6 degrees of freedom, 3 rotational degrees of freedom and 1 translational and 1 magnifying factor (zoom factor). FIG. 42 illustrates the 5 degrees of freedom of a C-arm machine 2900. Uniquely determining each of the 5 parameters is required for accurate 3-D reconstruction. Translation and zoom factors can be obtained by the method explained herein where rotational degrees of freedom can be uniquely determined by analyzing the angle information from the live-feed video data (as seen in FIG. 21). Alternately, it can also be measured using optical or magnetic sensors to track the motion of C-arm 2900. Information regarding the position of C-arm machine 2900 can also be obtained from within the motors attached to it, if one had access to the electrical signals sent to the motors.

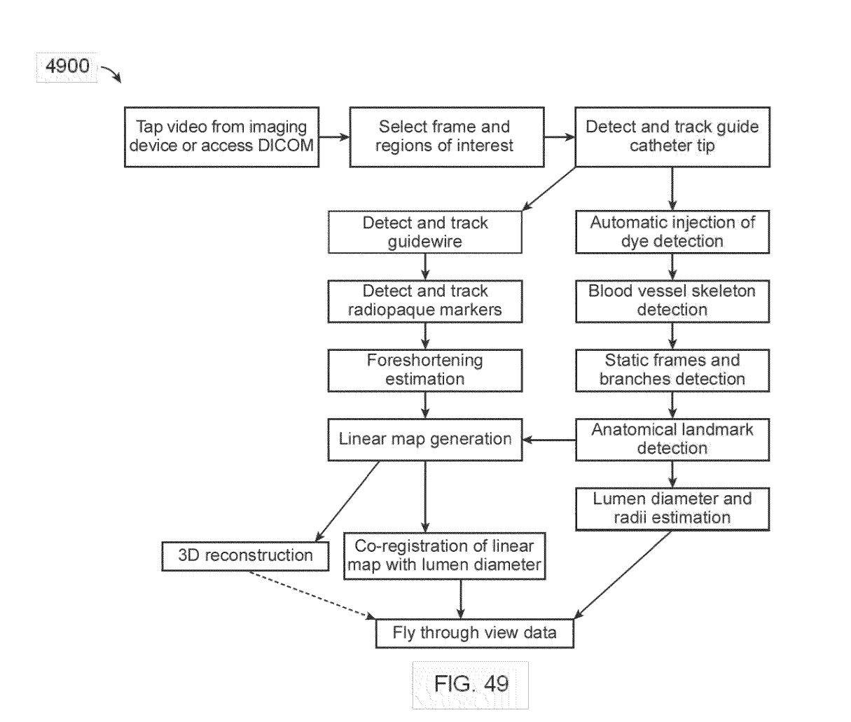

An example of an overall summary of the Analysis mode of operation is illustrated in the block diagram 4900 of FIG. 49 which illustrates various algorithms described herein involved.

Guidance Mode of Operation

Assuming that a co-registered and linearized map already exists, guidance mode of operation helps in guiding treatment devices to the lesion location. In one embodiment, images during the guidance mode of operation are in the same C-arm projection angle as it was at the time of linearized map creation. In such a case, mapping from image coordinates to linearized map coordinates is trivial and it involves marker detection and motion compensation techniques as discussed in previous sections. In another embodiment, the change in projection angle is significant. In such a case, a 3-D reconstructed view of the vessel path is used to map the linearized map generated from the previous angle to the present angle. After transformation, all the steps involved in the previous embodiment are used in this one as well. In yet another embodiment, guidance mode of operation when an accurate 3-D reconstruction is unavailable is done with the help of markers present in the treatment device. In such a case, these markers are used for linearizing the vessel in the new projection angle. Linearizing in the new angle automatically co-registers the map with the previously generated linearized map and thus the treatment device can be guided accurately to the lesion. An example of mapping the position of a catheter 1700 with electrodes and balloon markers is shown positioned along the linear map 1702 in FIG. 17.

This display is shown in real time. As the physician inserts or retracts the catheter, image processing algorithms run in real time to identify the reference points on the catheter, and map the position of the catheter in a linear display. The same linear display also shows the lumen profile. In one embodiment, the lumen dimension profile is estimated before the catheter is inserted. In another embodiment, the lumen dimension is measured with the same catheter using the active electrodes at the distal end of the catheter. As the catheter is advanced, the lumen dimension is measured and the profile is created on the fly.

While the disclosed invention is shown to work with X-ray images, the same concepts can be extended to other imaging methods such as MR, PET, SPECT, ultrasound, infrared, endoscopy, etc. in which the some features of the instrument inserted into a lumen are distinctly visible.

Lesion Delineators

Lesion delineators are the points along the linearized map generated which correspond to medically relevant locations in an image which represent a lesion. Points A and B (as illustrated in FIG. 16) are the points which represent the proximal and distal end of the lesion respectively. The linearized view when co-registered with lumen diameter measurement is capable of detecting this automatically. But the decision of selecting these points interactively during an intervention is left to the judgment of an interventionalist. M is the point of the co-registered plot which correspond the point where the lumen diameter is the least. R is the point on the co-registered plot whose diameter may be taken as a reference for selecting an appropriate stent diameter. The distance between A and B also helps in selecting the appropriate length of the stent. Points A, B, M, and R are collectively known as lesion delineators. This is output number 2 as seen in FIG. 18.

Method for Placement and Post Dilatation of Bio-Absorbable Stents

The placement of stents is achieved through angiographic guidance. In this method the user relies on a live X-ray image of radiopaque markers on a device (stent catheter) on one display coupled with a static image (referred of the angiogram) of the same vessel as a road map. The static image or angiogram is contrast enhanced and shows the lesion (blockage) where the stent needs to be placed. The stent delivery catheter is advanced to the point of interest and positioned in place by visually estimating the stenotic region on the previously-obtained still angiographic image. The angiographic images are 2D and suffer from foreshortening effects and are subject to gross errors in case of tortuous vessel. This is a very well-known phenomenon and the physician has to rely only on his or her own experience and skill. This technique can render the stents being geographically misplaced longitudinally (i.e., the expanded stent does not cover the entire blockage). For example the STLLR study (1557 patients) showed ˜48% of the stents are longitudinally misplaced.

To mitigate this issues methods and systems to guide a therapy device to the region of interest have been disclosed in U.S. Pat. No. 8,374,689, which is incorporated herein by reference in its entirety. Bioabsorbable stents are comprised of non-radio-opaque polymeric materials such as PLA/PGA. Since the stents are not visible under the X-ray there are small platinum (Pt) dots placed on the stent edges to demarcate them. However, visibility of the stent edges is poor as the Pt dots are barely visible and additionally they are out of plane. Due to this it is important to have a confirmed ‘stent landing zone’.

Secondly, after stent deployment, if you have to post-dilate you can't see the stent and its edges. This may lead to edge dissections if post dilation balloon is improperly positioned. It is further noted that because of a polymer stent, ‘post dilation’ is necessary most of the time therefore warranting a need for a technology to guide the placement of subsequent devices to the landing zone.

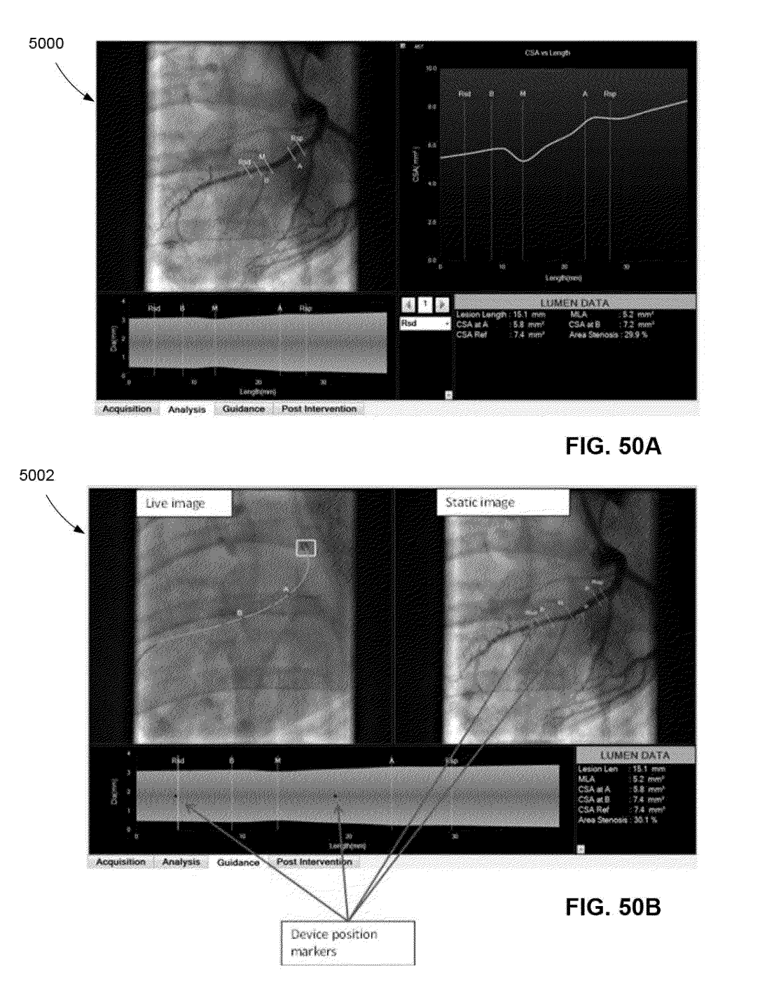

Using techniques described in U.S. Pat. No. 8,374,689, it is possible for the user to demarcate the lesion which is then superimposed on the static angiogram and the live angiogram, as shown in FIGS. 50A and 50B. As the bioabsorbable stent catheter is being deployed, the radiopaque markers are tracked in each image frame and the position superimposed on the static angiogram, as shown in image 5000, thus helping with the stent deployment. Furthermore since the positions A and B remain superimposed on both live and Static angiograms, as shown in image 5002, it is relatively easy for the user to come in with a post dilation balloon catheter and position it at the correct location thus avoiding procedural complications such as stent edge dissection.

The applications of the devices and methods discussed above are not limited to the examples and illustrations described herein but may include any number of further treatment applications. Moreover, such devices and methods may be applied to various other treatment sites within the body. Modification of the above-described assemblies and methods for carrying out the invention, combinations between different variations as practicable, and variations of aspects of the invention that are obvious to those of skill in the art are intended to be within the scope of the claims.

Example of Workflow for Ivus Co-Registration

In a first clinical step, the user initiates the co-registration session. At this point, two possible options for user interaction may occur. In the first option, the user provides a reference to the GC tip from images provided by imaging module. In the second option, the user does not have to provide a reference to the GC tip. The imaging module automatically detects the tip of the GC by automatically detecting the first angiogram that is performed after initiation. This angiogram is then analyzed to determine the position of the GC tip. The GC tip is then tracked automatically across all frames.

The algorithm detects the tip section of the guidewire. This section is the most prominently visible feature visible in the image, and is detected with good robustness. Once the positions of the two-ends of the guidewire are reliably found, the intermediate section of the guidewire is detected and tracked. The algorithm used to detect the guidewire is inherently robust. Image processing algorithms selectively extract features that can discriminate guidewire shaped objects, thus allowing for effective detection. Further, there are other mechanisms built in to ensure robust detection of the entire guide wire even in difficult situations where the guidewire is not completely visible. These include narrowing down of segments of the frame to be analyzed by using the GC tip and the detected angiogram, using past fluoro images captured at the same phase of the heart cycle, applying appropriate models and physical constraints on the trajectory of the guidewire, and selectively looking for objects that are consistent with the periodic movement due to heartbeat.

In a second clinical step, the user may perform the angiogram. At the time of an angiogram, injection of dye is automatically detected when the artery gets lit up. This detection triggers the algorithm pertaining to analysis of artery paths. Anatomical assessment is performed on the angiogram and distinct landmarks including branching points and lumen profile in the artery are identified across different phases of the heart-beat. These landmarks serve as anchor points around which a correspondence between points on the artery across phases of the heart are obtained. From multiple angiographic images, the one that best illuminates the arteries and branches is selected and communicated to the Master Client, e.g., iLab™ Ultrasound Imaging System (Boston Scientific Corp., MA) as a reference angiogram (referred to as RXI). Angiographic images corresponding to all phases of the heart cycle are stored internally in the imaging module for future reference.

In a third clinical step, the IVUS catheter may be inserted in the artery. When an IVUS catheter is inserted into the artery, the radiopaque transducer of the IVUS as well as catheter sheath marker (together referred to as IVUS markers) is detected and tracked across frames. Detection of the guidewire significantly helps in reducing the search-space for IVUS marker detection. Any resultant translation because of the movement of C-arm or the patient table and changes in scale of the image is estimated and accounted for in tracking all the objects of interest.

In a fourth clinical step, the IVUS catheter may be inserted into the artery. When the IVUS pullback starts, each recorded frame is mapped to a corresponding reference angiographic frame (RXI) based on the phase of the heartbeat. The point correspondence between that phase of the heartbeat and the phase that was provided to iLAB is already known. This is used to map the position of the IVUS transducer on to the RXI. This mapping is further refined based on the knowledge of the speed of the IVUS, and using raw results from past and future frames. Once the work in progress that estimates foreshortening during IVUS insertion is completed, this would be an additional factor taken into account for refining the mapping. The final refined mapping is used as the co-registered location for the IVUS transducer. The IVUS images obtained in time domain are matched with the time corresponding time domain transducer positions on the co-registered RXI.

What is claimed is:

1. A method of compensating for motion of a body lumen during co-registration, comprising:
positioning an elongate instrument having one or more markers within the body lumen to be mapped;
imaging the elongate instrument and the one or more markers along the elongate instrument within the body lumen;
tracking the one or more markers across multiple imaged frames;
referencing the one or more markers across the multiple imaged frames relative to at least one reference point separate from the elongate instrument;
matching predetermined reference points along the elongate instrument between the multiple imaged frames;
compensating for motion of the one or more markers based on the reference points along the elongate instrument which are matched between the multiple imaged frames; and
determining corresponding locations of the one or more markers on a reference image frame.

2. The method of claim 1 further comprising creating a linear map of the body lumen from the multiple imaged frames.

3. The method of claim 1 wherein referencing comprises referencing the one or more markers relative to at least one anatomical reference point.

4. The method of claim 3 wherein referencing comprises identifying at least one branching point or lumen profile.

5. The method of claim 4 wherein identifying comprises identifying the at least one branching point or lumen profile across the multiple imaged frames.

6. The method of claim 1 wherein referencing comprises referencing the one or more markers relative to at least one geometrical landmark.

7. The method of claim 1 wherein referencing comprises referencing the one or more markers relative to at least one reference point separate from the elongate instrument and positioned upon a patient.

8. The method of claim 1 wherein determining corresponding locations uses a priori knowledge of a movement pattern of the elongate instrument.

9. The method of claim 1 wherein the elongate instrument comprises a guidewire or catheter.

10. The method of claim 1 wherein tracking comprises tracking the one or more markers across multiple imaged frames which correspond to neighboring phases of a heart cycle.

11. The method of claim 1 wherein tracking comprises tracking the one or more markers across multiple imaged frames which correspond to movement resulting from breathing.

12. The method of claim 1 wherein the motion of the body lumen is associated with movements from a beating heart or breathing of a patient.

13. The method of claim 1 wherein the motion of the body lumen is associated with movements of a patient, motion of a platform upon which the patient is positioned, or motion of a C-arm relative to the patient.

14. The method of claim 1 wherein determining further comprises selecting past frames and/or future frames to refine the corresponding locations on the reference image frame.

15. The method of claim 1 further comprising compensating for motion artifacts by compensating for motion between a current fluoroscopic image to be co-registered and an angiographic image corresponding to a same phase of a heart cycle as the fluoroscopic image, and
compensating for motion between the angiographic image of the same phase and a reference angiographic image.

16. The method of claim 1 further comprising enhancing an image for each pixel of the elongate instrument in the multiple imaged frames after imaging the elongate instrument and the one or more markers.

17. The method of claim 1 wherein the one or more markers comprise a subset of the region of interest in any single frame.

18. The method of claim 1 wherein the one or more markers comprise electrodes, radio-opaque markers, or one or more stents.

19. The method of claim 1 wherein the plurality of markers are spaced apart from one another at known distances.

20. The method of claim 1 further comprising injecting a dye into the body lumen during imaging.

21. The method of claim 1 wherein the body lumen comprises a blood vessel.

22. A method for determining the translation of an elongate instrument from multiple two-dimensional images of a moving body lumen, comprising:
positioning an elongate instrument having one or more markers within the body lumen to be mapped;
imaging the elongate instrument and the one or more markers along the elongate instrument within the body lumen;
tracking the one or more markers across multiple imaged frames;
matching predetermined reference points along the elongate instrument between the multiple imaged frames;
compensating for motion of the one or more markers based on the reference points along the elongate instrument which are matched between the multiple imaged frames, where the motion results from the effect of movement of the body lumen on the elongate instrument; and,
determining corresponding locations of the one or more markers on a reference image frame.

23. The method of claim 22 wherein the movement of the body lumen is associated with movements from a beating heart or breathing of a patient.

24. The method of claim 22 wherein the movement of the body lumen is associated with movements of a patient, motion of a platform upon which the patient is positioned, or motion of a C-arm relative to the patient.

25. The method of claim 22 wherein determining comprises superimposing a translation of the elongate instrument and one or more markers upon a stationary image of the body lumen.

26. The method of claim 22 further comprising creating a linear map of the body lumen from the multiple imaged frames.

27. The method of claim 22 further comprising enhancing an image of the elongate instrument in the multiple imaged frames after imaging the elongate instrument and the one or more markers.

28. The method of claim 22 wherein imaging the elongate instrument comprises moving the elongate instrument through the body lumen while imaging.

29. The method of claim 21 wherein tracking the one or more markers further comprises detecting and tracking the elongate instrument across the multiple imaged frames.

30. The method of claim 22 wherein the elongate instrument comprises a guidewire or catheter.

31. The method of claim 22 wherein the plurality of markers comprise electrodes, radio-opaque markers, or one or more stents.

32. The method of claim 22 further comprising co-registering one or more locations along the linear map with one or more corresponding landmarks.

33. The method of claim 22 wherein the body lumen comprises a blood vessel.

34. A method of selecting a vessel of interest, comprising:
positioning an elongate instrument within a vessel of interest;
tracking a position of at least a subset of the elongate instrument;
injecting a dye within the vessel of interest;
selecting at least one angiographic image;
processing the at least one reference angiographic image to segment a network of branches illuminated by the dye;
matching the tracked position of the at least a subset of the elongate instrument to the at least one processed angiographic image; and
selecting a vessel corresponding to the best matched segmented part of the network of branches.

35. The method of claim 34 wherein selecting comprises automatically selecting the at least one angiographic image.

36. The method of claim 34 wherein selecting comprises manually selecting the at least one angiographic image.

37. The method of claim 34 wherein the elongate instrument comprises a guidewire, catheter or at least one stent.

38. The method of claim 34 wherein matching comprises selecting a vessel branch which closely represents a shape and/or profile of the elongate instrument.

39. The method of claim 34 wherein the vessel of interest comprises a blood vessel.

40. A method of selecting a reference angiogram, comprising:
injecting a dye into a network of vessels while imaging the network of vessels over multiple image frames;
measuring at least one of:
a degree of contrast present in each of the image frames;
an extent of a vessel path highlighted in each of the image frames;
a length of each branch highlighted in the network of vessels in each of the image frames; and
determining an optimal image frame based on the at least one of degree of contrast, extent of the vessel path, and length of each branch.

41. A method of estimating a translation or zoom factor of an image, comprising:
recording multiple image frames of a vessel of interest of a patient;
analyzing a previous image frame with respect to a current frame;
calculating a metric value between pixels of each image frame; and
selecting an image frame having a minimum metric value,
wherein the translation or zoom is caused by at least one of a heartbeat of the patient, breathing of the patient, change in camera position relative to the patient, movement of a table upon which the patient is positioned, or movement of the patient.

42. The method of claim 41 wherein calculating a metric value comprises calculating a sum of squared differences or a sum of absolute differences between the pixels of each image frame.

43. The method of claim 41 further comprising differentiating between a rotation and a translation or zoom factor.
